(19) United States
(12) Patent Application Publication
(10) Pub. No.: US 2009/0214519 A1
(43) Pub. Date: Aug. 27, 2009

(54) Compositions and Methods for Treating Inflammation

(75) Inventors: Lieping Chen, Sparks Glencoe, MD (US); Koji Tamada, Lutherville, MD (US)

Correspondence Address: EDWARDS ANGELL PALMER & DODGE LLP, P.O. BOX 55874, BOSTON, MA, 02205, US

(73) Assignee: The John Hopkins University, Baltimore, MD (US)

(21) Appl. No.: 12/083,119

(22) PCT Filed: Oct. 4, 2006
(86) PCT No.: PCT/US2006/039072

§ 371 Date: Jan. 12, 2009

Related U.S. Application Data
(60) Provisional application No. 60/723,521, filed on Oct. 4, 2005.
(60) Provisional application No. 60/723,521, filed on Oct. 4, 2005.

(51) Int. Cl. A61K 39/395; G01N 33/68; A01K 67/00; A61P 29/00

(52) U.S. Cl. 424/130.1; 436/86; 800/13

(57) ABSTRACT

The present invention relates to compositions to treat inflammation (LIGHT pathway) related disorders, and specifically liver inflammation or hepatitis. The invention also relates to methods treating LIGHT pathway related disorders. The invention further relates to kits for treating LIGHT pathway related disorders in a subject. The invention further relates to methods of identifying novel treatments for treating LIGHT pathway related disorders in a subject.

### RELATED APPLICATIONS

This application claims priority to U.S. Provisional Application No. 60/723,521 filed Oct. 4, 2005, entitled, “Novel Method to Treat Hepatitis,” which is hereby incorporated by reference in its entirety.

### GOVERNMENT SUPPORT

This work was supported by the National Institutes of Health. The government may have certain rights in the invention.

### BACKGROUND

Hepatitis, which is an international health concern is inflammation of the liver. Several different viruses cause viral hepatitis caused by hepatitis A, B, C, D, and E viruses. Each of the viruses cause acute, or short-term, viral hepatitis. The hepatitis B, C, and D viruses can also cause chronic hepatitis, in which the infection is prolonged, sometimes lifelong. Hepatitis may also be caused by an auto-immune reaction which causes the liver to become inflamed. The disease is usually quite serious and chronic and, if not treated, gets worse over time leading to cirrhosis (scarring and hardening) of the liver and eventually liver failure.

Molecules belonging to the TNF superfamily play an integral role in the regulation of innate and adaptive immunity, as well as contributing to inflammatory responses through their effects on nonhematopoietic cells (1). LIGHT (homologous to lymphotoxin, exhibits inducible expression, and competes with HSV glycoprotein D for herpes virus entry mediator, a receptor expressed by T lymphocytes) is a recently identified type II transmembrane glycoprotein of the TNF ligand superfamily (2). LIGHT is expressed on immature DCs and activated T cells (2, 3) and binds to 3 distinct receptors, herpes virus entry mediator (HVEM), lymphotoxin-β receptor (LTβR), and decoy receptor 3/TR6 (2, 4). Upon binding to HVEM, LIGHT costimulates T cells and accelerates proliferation and cytokine production (3, 5). Gene targeting of LIGHT results in impaired T cell immunity and a compromised allograft rejection (6-8). Thus, there is ample evidence indicating a role of LIGHT in the adaptive immune system.

Liver inflammation is mediated by immune responses to hepatocytes following liver-tropic pathogen infections or by pathogenic autoreactivity. Exploration of molecular and cellular mechanisms underlying liver inflammation is to treat acute hepatitis and to prevent liver cirrhosis and hepatocellular carcinoma following chronic hepatitis. A number of regulators have been implicated in both human and experimental hepatitis, including TNF superfamily molecules such as TNF-α and FasL (17). Thus, there is a need in the art for methods of treating inflammation, especially liver inflammation and hepatitis.

### BRIEF SUMMARY OF THE INVENTION

This invention is based, in part, on the discovery that blocking the interaction between LIGHT and LTβR leads to a decreased inflammatory response. The present invention provides novel compositions, methods, and kits to treat inflammation, particularly liver inflammation. The invention further provides methods of identifying novel treatments for treating inflammation in a subject.

Provided herein, according to one aspect, are methods of modulating an interaction between LIGHT and LTβR comprising administering a LIGHT pathway protein modulator.

Provided herein, according to one aspect, are methods inflammation in a mammal, comprising modulating the LIGHT pathway protein signaling wherein disrupting an interaction between LIGHT and LTβR modulates inflammation of the a cell.

Provided herein, according to one aspect, are methods for the treatment and/or prophylaxis of a condition characterized by aberrant or otherwise unwanted inflammation in a subject, comprising modulating the LIGHT pathway protein signaling wherein disrupting an interaction between LIGHT and LTβR modulates inflammation of the a cell.

In one embodiment, the cell is one or more of a liver cell, a cartilage cell, a digestive tract cell, neuronal cell, a pancreatic cell, a lung cell, bone tissue cell, a spleen cell, heart cell, kidney cell, a testis cell, or an intestinal tract cell.

In another embodiment, the protein comprises one or more LIGHT pathway family proteins.

In one embodiment, the LIGHT pathway family protein comprises LIGHT or LTβR.

In one embodiment, wherein inflammation is reduced or alleviated by disrupting an interaction between LIGHT and LTβR modulates inflammation of the a cell.

In another embodiment, the condition is one or more of liver inflammation, hepatitis (autoimmune and pathogen induced), arthrosclerosis, arthritis, IgA nephropathy, inflammatory bowel disease, liver cirrhosis, and hepocellular carcinoma.

In one embodiment, the modulation comprises contacting the cell with a compound that modulates an interaction between LIGHT and LTβR.

In one embodiment, the modulation is a down-regulation of soluble LIGHT.

In one embodiment, the modulation is down-regulation of LIGHT protein pathway signaling.

In another embodiment, the inflammation is modulated in vivo.

In one embodiment, the inflammation is modulated in vitro.

Provided herein, according to one aspect are pharmaceutical compositions comprising a pharmaceutically effective amount of a LIGHT pathway modulator effective to treat, prevent, ameliorate, reduce or alleviate a LIGHT pathway related disorder or symptoms thereof and a pharmaceutically acceptable excipient. In one embodiment, symptoms include, for example pain, jaundice, fever, fatigue, vomiting, nausea, diarrhea, appetite loss, hepatomegaly, liver cirrhosis, hepatocellular carcinoma, or hepatitis. One or more of these being alleviated may indicate efficacy of treatment as described herein.

In one embodiment, the LIGHT pathway modulator is selected from one or more of a small molecule, an anti-LIGHT pathway antibody, an antigen-binding fragment of an anti-LIGHT pathway antibody, a polypeptide, a peptidomimetic, a nucleic acid encoding a peptide, or an organic molecule.

In one embodiment, the LIGHT pathway related disorder comprises one or more of liver inflammation, hepatitis (autoimmune and pathogen induced), arthrosclerosis, arthritis, IgA nephropathy, inflammatory bowel disease, liver cirrhosis, and hepocellular carcinoma.

Provided herein, according to one aspect, are methods to treat, prevent, ameliorate, reduce or alleviate a LIGHT pathway related disorder or symptoms thereof, comprising: administering to a subject in need thereof a composition comprising a pharmaceutically effective amount of a LIGHT pathway modulator.

In one embodiment, the LIGHT pathway related disorder comprises one or more of liver inflammation, hepatitis (autoimmune and pathogen induced), arthrosclerosis, arthritis, IgA nephropathy, inflammatory bowel disease, liver cirrhosis, and hepocellular carcinoma.

In another embodiment, the LIGHT pathway modulator is one or more of a small molecule, an anti-LIGHT pathway antibody, an antigen-binding fragment of an anti-LIGHT pathway antibody, a polypeptide, a peptidomimetic, a nucleic acid encoding a peptide, or an organic molecule.

In another embodiment, the LIGHT pathway modulators is an anti-LTβR antibody or a fragment or variant thereof.

In one embodiment, the LIGHT pathway modulators is a LLTB1,a LIGHT, and/or a B7-H4 antibody or fragment thereof.

In one embodiment, the LIGHT pathway modulator is administered prophylactically to a subject at risk of being afflicted a LIGHT pathway related disorder.

In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

In another embodiment, the composition is administered to the subject orally, intravenously, intrathecally or epidurally, intramuscularly, subcutaneously, perineurally, intradermally, topically or transcutaneously.

In another embodiment, the subject is a mammal.

In one embodiment, the subject is a human.

In one embodiment, a LIGHT pathway related disorder or symptom thereof is indicated by alleviation of pain, and/or alleviation of hepatitis.

In one embodiment, the methods may further comprise obtaining the LIGHT pathway modulator.

Provided herein, according to one aspect, are methods for identifying lead compounds for a pharmacological agent useful in the treatment of a LIGHT pathway related disorder comprising contacting a cell stimulated with concanavalin A with a test compound, and measuring LIGHT pathway activation or inflammation.

Provided herein, according to one aspect, are methods for identifying lead compounds for a pharmacological agent useful in the treatment of a LIGHT pathway related disorder comprising contacting a cell stimulated with concanavalin A with a test compound, and measuring soluble form of LIGHT.

In one embodiment, measuring soluble form of LIGHT is by one or more of measuring protein levels of one soluble LIGHT.

In another embodiment, the test compounds is one or more of a peptide, a small molecule, an antibody or fragment thereof, and nucleic acid or a library thereof.

Provided herein, according to one aspect, are kits comprising, for example, a LIGHT pathway modulator and a pharmaceutically acceptable carrier and instructions for use.

Provided herein, according to one aspect, are transgenic non-human animals comprising a Y173F LIGHT protein or a fragment or variant thereof.

Provided herein, according to one aspect, are transgenic non-human animals comprising a LIGHTΔL protein or a fragment or variant thereof.

Provided herein, according to one aspect, are uses of a transgenic animal according to the method described herein, to test therapeutic agents.

Provided herein, according to one aspect, are methods for screening a therapeutic agent to treat, prevent, ameliorate, reduce or alleviate a LIGHT pathway related disorder or symptoms thereof, comprising administering a test agent to an animal, and measuring modulation of one or more of inflammation, clearance of bacteria, liver inflammation, infiltration of inflammatory cells, and/or hepatocyte necrosis.

In one embodiment, the animal is a mouse.

In another embodiment, the mouse is one or more of a normal mouse or a mouse expressing one or more of a Y173F LIGHT or a LIGHTΔL protein.

In one embodiment, the methods may further comprise inducing hepatitis in the animal.

In one embodiment, hepatitis is induce by treatment with concanavalian A, and/or listeria monocytogenes as well as other methods known in the art to induce either hepatitis or liver inflammation.

In another embodiment, a decrease inflammation or soluble LIGHT indicate that the test agent may be useful in treating a LIGHT pathway disorder.

Provided herein, according to one aspect, are methods of treating inflammation in a mammal, comprising: (a) identifying a mammal with, or at risk of developing, inflammation, wherein the cells of the cancer are identified as expressing soluble LIGHT; and (b) administering to the subject a compound comprising an agent that interferes with an interaction between LIGHT and a LTβR.

Provided herein, according to one aspect, are methods of diagnosing inflammation comprising, detecting the presence of soluble LIGHT in a sample from a subject.

In one embodiment, the inflammation comprises one or more of scleroderma or hepatitis.

In one embodiment, the sample comprises one or more of a blood sample, a bronchioalveolar lavage sample, and/or a sputum sample, as well as other biological samples wherein a LIGHT pathway protein or soluble LIGHT may be detected.

Provided herein, according to one aspect, are methods for identifying lead compounds for a pharmacological agent useful in the treatment of a LIGHT pathway related disorder comprising contacting a cell hepatitis model with a test compound, and measuring soluble form of LIGHT.

In another embodiment, measuring soluble form of LIGHT is by one or more of measuring protein levels of one soluble LIGHT.

In one embodiment, the test compounds is one or more of a peptide, a small molecule, an antibody or fragment thereof, and nucleic acid or a library thereof.

Other embodiments of the invention are disclosed infra.

### BRIEF DESCRIPTION OF THE DRAWINGS

FIG. 1 shows the pathogenic role of upregulated LIGHT in hepatitis. (A) BALB/c mice were injected i.v. with 25 mg/kg ConA. At the indicated time points, the mice were sacrificed, and total RNA was extracted from liver and spleen. LIGHT, HVEM, LTβR, and GAPDH expression was examined by Northern blot analysis. (B and C) Wild-type (open circles) and LIGHT-deficient (filled circles) mice were injected i.v. with 30 mg/kg ConA. The survival (B) and serum ALT levels (C) of recipient mice were monitored. Sera were collected 18 hours after ConA injection, and the ALT levels were measured as described in Methods. SF, Sigma-Frankel. *p=0.003.

FIG. 2 demonstrates the role of the soluble form of LIGHT in liver inflammation. (A) B6 mice were injected i.v. with 30 mg/kg of ConA. At the indicated time points, serum was collected from the recipient mice and measured for soluble LIGHT concentration by LIGHT-specific ELISA. (B and C) B6 mice were injected with a sublethal dose of ConA (12.5 mg/kg) alone (filled circles, n=13) or together with 20 μg plasmids encoding control pcDNA3.1 (open circles, n=13), wild-type LIGHT (filled squares, n=11), or LIGHTL (open squares, n=10) by hydrodynamic injection technique. Survival of mice (B) and liver sections stained with H&E 18 hours after injection (C) were examined. N, necrotic area. *P=0.3, **P=0.033 between the groups by log-rank test. (D) BALB/c mice were injected i.p. with 50 μg of soluble LIGHT-flag fusion protein or control protein. One hour later, the mice were injected i.v. with 25 mg/kg ConA, and serum ALT levels were measured 6 hours later. One representative result from 3 independent experiments is shown as mean±SD.

FIG. 3 shows that LTβR is necessary and sufficient for LIGHT-mediated hepatitis. (A) B6 mice were injected with either empty vector or plasmid DNA encoding wild-type LIGHT or Y173F mutant by hydrodynamic method in combination with a sublethal dose of ConA (12.5 mg/kg). ALT levels were measured 18 hours after injection. One representative result from 2 independent experiments is shown as mean±SD of 5 mice per group. (B) B6 mice were injected i.v. with 30 mg/kg of ConA together with 100 μg of control rat Ig (open circles, n=12), anti-LTβR (filled circles, n=10), or anti-HVEM mAb (filled squares, n=9). The survival was monitored thereafter. (C) B6 mice were injected with either empty vector or plasmid DNA encoding LIGHT by hydrodynamic method in combination with a sublethal dose of ConA (12.5-mg/kg). The mice were treated i.p. with 100 μg of the indicated Abs 2 hours before the plasmid injections. After 18 hours, serum ALT levels were measured. One representative result from 2 independent experiments is shown as mean±SD of 5 mice per group. hIg, control hamster Ig; rIg, control rat Ig.

FIG. 4 shows the role of NKT cells in the production of soluble LIGHT in ConA-induced hepatitis. (A and B) B6 mice were treated i.p. with 500 μg of either control mouse IgG or anti-NK1.1 mAb (PK136) on days 0 and 3 (A). Similarly, B6 mice were treated i.p. with 30 μg of control rabbit IgG or anti-asialo GM1 (ASGM1) on days 0 and 3 (B). On day 4, each group was injected i.v. with 30 mg/kg of ConA, and mouse sera were collected 1 hour later. The amounts of soluble LIGHT were measured by ELISA. *P=0.02. (C) CD1d-deficient mice were injected with 20 μg of control vector or LIGHT-encoding plasmid by hydrodynamic injection in combination with a sublethal dose of ConA (12.5 mg/kg). Mouse sera were collected 18 hours later, and ALT levels were measured. **P=0.002.

FIG. 5 shows the blockade of LIGHT-LTβR interaction as a treatment for L. monocytogenes-induced hepatitis. (A) B6 mice (open circles, n=21) and LIGHT-deficient mice (filled triangles, n=21) were injected i.p. with L. monocytogenes (2×LD50 per mouse), and their survival was monitored. P=0.007 between the groups. (B) B6 mice were injected i.v. with 100 μg of control IgG (open circles, n=10) or anti-LTβR mAb (filled circles, n=11). At the same time, the mice were infected with L. monocytogenes (2×LD50 per mouse) by i.p. injection. Survival of mice was monitored thereafter. P=0.03 between the groups. (C) As in B, B6 mice were infected with L. monocytogenes and treated with either anti-LTβR mAb or control IgG. Three days after infection, liver sections were prepared and stained with H&E.

### DETAILED DESCRIPTION

This invention is based, in part, on the discovery that blocking the interaction between LIGHT and LTβR leads to decreased inflammatory response. The present invention provides novel compositions, methods, and kits to treat inflammation, particularly liver inflammation. The invention further provides methods of identifying novel treatments for treating inflammation in a subject.

“Agonist,” as used herein refers to a compound or composition capable of combining with (e.g., binding to, interacting with) receptors to initiate pharmacological actions.

Pharmaceutically acceptable refers to, for example, compounds, materials, compositions, and/or dosage forms which are suitable for use in contact with the tissues of human beings and animals without excessive toxicity, irritation, allergic response, or other problems or complications, commensurate with a reasonable benefit/risk ratio.

Pharmaceutically acceptable salts refer to, for example, derivatives of the disclosed compounds wherein the compounds are modified by making at least one acid or base salt thereof, and includes inorganic and organic salts.

An effective antagonistic amount of modulator refers to an amount that effectively attenuates (e.g. blocks, inhibits, prevents, or competes with) the activity of the protein.

A therapeutically effective amount of a LIGHT pathway composition refers to an amount that elicits alleviation or lessening of at least one symptom of pain upon administration to a subject in need thereof.

The following terms encompass polypeptides that are identified in Genbank by the following designations, as well as polypeptides that are at least about 70% identical to polypeptides identified in Genbank by these designations as described infra. In alternative embodiments, these terms encompass polypeptides identified in Genbank by these designations and polypeptides sharing at least about 80, 90, 95, 96, 97, 98, or 99% identity.

“LIGHT” (TNFSF14) is structurally and functionally an integral member of the immediate TNF family, defined by a close structural homology and a communal pattern of receptor-ligand pairing with lymphotoxin-αβ (LTαβ), LTα, and Fas ligand (Mauri D N, et al. LIGHT, a new member of the TNF superfamily, and lymphotoxin α are ligands for herpesvirus entry mediator. Immunity. 1998; 8:21-30; Yu K Y, et al. A newly identified member of tumor necrosis factor receptor superfamily (TR6) suppresses LIGHT-mediated apoptosis. J Biol Chem. 1999; 274:13733-13736). LIGHT is a type II transmembrane protein, produced by activated T cells and immature dendritic cells, that signals through two distinct cellular receptors: the herpesvirus entry mediator (HVEM), which is expressed prominently on T cells, and the LTβ receptor (LTβR), which is expressed on stromal cells but absent from lymphocytes. LIGHT has been proposed to mediate T cell activation, survival, or death, and also—by analogy with LTαβ—to help organize lymphoid tissues (Fu Y-X, Chaplin D. Development and maturation of secondary lymphoid tissues. Annu Rev Immunol. 1999; 17:399-433). Indeed, results in tissue culture models appear to support a role for LIGHT in T cell activation (Harrop J A, et al. Herpesvirus entry mediator ligand (HVEM-L), a novel ligand for HVEM/TR2, stimulates proliferation of T cells and inhibits HT29 cell growth. J Biol Chem. 1998; 273:27548-27556; Tamada K, et al. LIGHT, a TNF-like molecule, costimulates T cell proliferation and is required for dendritic cell-mediated allogeneic T cell response. J Immunol. 2000; 164:4105-4110), although the responses of T cells to LIGHT signaling in vitro are rather subtle. The phenotype of mice expressing ectopic LIGHT is anything but subtle.

As used here, “LIGHT pathway protein,” refers to a protein involved in the signaling or activation or downstream target of soluble LIGHT.

A “LIGHT pathway modulator” is either an inhibitor or an enhancer of a LIGHT protein family member. A “non-selective” LIGHT pathway protein modulator is an agent that modulates other LIGHT protein family members at the concentrations typically employed for LIGHT pathway modulation. A “selective” modulator significantly modulates one or more of the normal functions of a pathway protein family member at a concentration at which other pathway proteins are not significantly modulated. A modulator “acts directly on” a LIGHT pathway protein family member when the modulator binds to the LIGHT pathway protein. A modulator “acts indirectly on a LIGHT pathway protein” when the modulator binds to a molecule other than the LIGHT pathway protein, which binding results in modulation of the protein.

As used herein, “interferes with an interaction between the LIGHT and LTβR” include, for example: (a) completely blocks a physical interaction between the LIGHT and LTβR such that there is substantially no physical interaction between the LIGHT and LTβR; or (b) modifies the physical interaction of the LIGHT and LTβR such that the physical interaction either does not deliver a signal or delivers a signal that does not substantially result in inflammation.

As used herein, a “functional fragment” of a LIGHT pathway protein is a fragment of the protein that is shorter than the full-length protein and has the ability retain its function.

A “modulator of an interaction between LIGHT and LTβR” is an agent that reduces or eliminates signaling between LIGHT and LTβR, as compared to that observed in the absence (or presence of a smaller amount) of the agent. A modulator of an interaction between LIGHT and LTβR can affect: (1) the expression; mRNA stability; or protein trafficking, modification (e.g., phosphorylation), or degradation of a protein family member, or (2) one or more of the normal functions of a protein family member, such the depolarization-induced inward current. A modulator the interaction between LIGHT and LTβR can be non-selective or selective.

An “enhancer of a LIGHT pathway protein” is an agent that increases, by any mechanism as compared to that observed in the absence (or presence of a smaller amount) of the agent. An enhancer of a protein can affect: (1) the expression; mRNA stability; or protein trafficking, modification (e.g., phosphorylation), or degradation of a protein; or (2) one or more of the normal functions of a protein. An enhancer of an protein can be non-selective or selective.

In one embodiment the present invention is directed to down regulating the functional level of LIGHT pathway to reduce or prevent or alleviate inflammation to a population of cells. However, it should nevertheless be understood that there are circumstances in which it is desirable to up-regulate the functional level of LIGHT pathway to induce inflammation in the cells. For example, one may seek to up regulate the functional level of LIGHT pathway in the context of a defined population of cells for a period of time sufficient to achieve a particular objective. However, once that objective has been achieved one would likely seek to down regulate the intracellular functional level of LIGHT pathway, to the extent that it is not transient, such that it is no longer over-expressed and the subject cells. In another example, one may identify certain disease conditions which are characterized by an over-expression or activation of the LIGHT pathway, e.g., liver inflammation, hepatitis (autoimmune and pathogen induced), arthrosclerosis, arthritis, IgA nephropathy, inflammatory bowel disease, liver cirrhosis, and hepocellular carcinoma. Where such an over stimulation of the LIGHT pathway or improper stimulation or activation of the LIGHT pathway situation exists, one may seek to down regulate the functional level of LIGHT pathway to end aberrant inflammation. Accordingly, down-regulation of cell LIGHT pathway signaling would be desirable as a therapeutic treatment. The present invention should therefore be understood to be directed to down regulating the LIGHT pathway signaling in order to introduce unique phenotypic properties to the population of cells and down-regulating a naturally or non-naturally induced state of LIGHT pathway over-expression.

LIGHT pathway related disorder or symptoms thereof, refers to inflammation, e.g., liver inflammation, hepatitis (autoimmune and pathogen induced), arthrosclerosis, arthritis, IgA nephropathy, inflammatory bowel disease, liver cirrhosis, and hepocellular carcinoma and associated symptoms.

As detailed above, reference to “modulating” LIGHT pathway signaling is a reference to either up regulating or down regulating the signaling though the pathway. Such modulation may be achieved by any suitable means and include, for example: (i) modulating absolute levels of the active or inactive forms of LIGHT pathway proteins (for example increasing or decreasing intracellular LIGHT pathway protein concentrations) such that either more or less of one or more of the LIGHT pathway proteins are available for activation and/or to interact with its downstream targets. (ii) Agonising or antagonising one or more LIGHT pathway proteins such that the functional effectiveness of any given LIGHT pathway protein molecule is either increased or decreased. For example, decreasing/increasing the half life of LIGHT pathway proteins may achieve a decrease in the overall level of LIGHT pathway activity without actually necessitating a decrease in the absolute intracellular concentration of LIGHT pathway. Similarly, the partial antagonism of one or more LIGHT pathway proteins, for example by coupling LIGHT pathway to a molecule that introduces some steric hindrance in relation to the binding of LIGHT pathway to its downstream targets, may act to reduce, although not necessarily eliminate, the effectiveness of LIGHT pathway signaling. Accordingly, this may provide a means of down-regulating LIGHT pathway functioning without necessarily down-regulating absolute concentrations of LIGHT pathway.

In terms of achieving the up or down-regulation of LIGHT pathway functioning, methods and techniques for achieving this objective would be well known to the person of skill in the art and include, for example: (i) introducing into a cell a nucleic acid molecule encoding LIGHT pathway or functional equivalent, derivative or analogue thereof in order to up-regulate the capacity of The cell to express LIGHT pathway. (ii) Introducing into a cell a proteinaceous or non-proteinaceous molecule which modulates transcriptional and/or translational regulation of a gene, wherein this gene may be a LIGHT pathway gene or functional portion thereof or some other gene which directly or indirectly modulates the expression of the LIGHT pathway gene. (iii) introducing into a cell the LIGHT pathway expression product (in either active or inactive form) or a functional derivative, homologue, analogue, equivalent or mimetic thereof. (iv) introducing a proteinaceous or non-proteinaceous molecule which functions as an antagonist to the LIGHT pathway expression product. (v) introducing a proteinaceous or non-proteinaceous molecule which functions as an agonist of the LIGHT pathway expression product. (vi) antibody to a LIGHT pathway protein (e.g., an anti-LTβR antibody or fragment or similar molecule, e.g., single-chain or portion thereof.); RNAi, siRNA, aptamer, or small molecule.

As used herein, the term “antibody” refers not only to whole antibody molecules, but also to antigen-binding fragments, e.g., Fab, F(ab′).sub.2, Fv, and single chain Fv (sFv) fragments. An sFv fragment is a single polypeptide chain that includes both the heavy and light chain variable regions of the antibody from which the sFv is derived. Such fragments can be produced, for example, as described in U.S. Pat. No. 4,642,334, which is incorporated herein by reference in its entirety. Also included are chimeric antibodies. Chimeric antibodies are recombinant antibodies comprising portions derived from more than one species; for example, the antigen binding regions (i.e., the complementarity determining regions (CDR)), of the antibody molecule can be derived from a mouse wild-type antibody molecule and framework and constant regions can be derived from a human antibody molecule or human antibody molecules.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

“Anti-inflammatory compounds” as used herein include compounds directed at blocking or reducing inflammation and analgesics directed to reducing pain, e.g., aspirin and other salicylate compounds, phenylpropionic acid derivatives such as Ibuprofen and Naproxin, Sulindac, phenyl butazone, corticosteroids, antimalarials such as chloroquine and hydroxychloroquine sulfate, and fenemates. For a thorough review of various drugs utilized in treating rheumatic diseases, reference is made to J. Hosp. Pharm., 36:622 (May 1979).

A “test agent” is any agent that can be screened in the prescreening or screening assays of the invention. The test agent can be any suitable composition, including a small molecule, peptide, or polypeptide.

The term “therapy,” as used herein, encompasses the treatment of an existing condition as well as preventative treatment (i.e., prophylaxis). Accordingly, “therapeutic” effects and applications include prophylactic effects and applications, respectively.

A used herein, the term “high risk” refers to an elevated risk as compared to that of an appropriate matched (e.g., for age, sex, etc.) control population.

“Nucleic acids,” as used herein, refers to nucleic acids that are isolated a natural source; prepared in vitro, using techniques such as PCR amplification or chemical synthesis; prepared in vivo, e.g., via recombinant DNA technology; or by any appropriate method. Nucleic acids may be of any shape (linear, circular, etc.) or topology (single-stranded, double-stranded, supercoiled, etc.). The term “nucleic acids” also includes without limitation nucleic acid derivatives such as peptide nucleic acids (PNA's) and polypeptide-nucleic acid conjugates; nucleic acids having at least one chemically modified sugar residue, backbone, internucleotide linkage, base, nucleoside, or nucleotide analog; as well as nucleic acids having chemically modified 5′ or 3′ ends; and nucleic acids having two or more of such modifications. Not all linkages in a nucleic acid need to be identical.

In general, the oligonucleotides may be single-stranded (ss) or double-stranded (ds) DNA or RNA, or conjugates (e.g., RNA molecules having 5′ and 3′ DNA “clamps”) or hybrids (e.g., RNA:DNA paired molecules), or derivatives (chemically modified forms thereof). However, single-stranded DNA is preferred, as DNA is often less labile than RNA. Similarly, chemical modifications that enhance an aptamer's specificity or stability are preferred.

Chemical modifications that may be incorporated into nucleic acids include, with neither limitation nor exclusivity, base modifications, sugar modifications, and backbone modifications. Base modifications: The base residues in aptamers may be other than naturally occurring bases (e.g., A, G, C, T, U, 5MC, and the like). Derivatives of purines and pyrimidines are known in the art; an exemplary but not exhaustive list includes aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine (5MC), N6-methyladenine, 7-methylguanine, 5-methylaminomethyluracil, 5-methoxyaminomethyl-2-thiouracil, beta-D-mannosylqueosine, 5-methoxyuracil, 2-methylthio-N-6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. In addition to nucleic acids that incorporate one or more of such base derivatives, nucleic acids having nucleotide residues that are devoid of a purine or a pyrimidine base may also be included in aptamers. Sugar modifications: The sugar residues in aptamers may be other than conventional ribose and deoxyribose residues. By way of non-limiting example, substitution at the 2′-position of the furanose residue enhances nuclease stability. An exemplary, but not exhaustive list, of modified sugar residues includes 2′ substituted sugars such as 2′-O-methyl-, 2′-O-alkyl, 2′-O-allyl, 2′-S-allyl, 2′-S-allyl, 2′-fluoro-, 2′-halo, or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and abasic nucleoside analogs such as methyl riboside, ethyl riboside or propylriboside.

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3-(and 4-) hydroxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Om), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters and is well known by one of skill in the art. For example, optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al. (1990) J. Mol. Biol. 215: 403-410. In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787).

The term “specific binding” is defined herein as the preferential binding of binding partners to another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

The term antibody, as used herein also includes antibody fragments either produced by the modification of whole antibodies or synthesized de novo using recombinant DNA methodologies, see for example, Fundamental Immunology, W. E. Paul, ed., Raven Press, N.Y. (1993), for a more detailed description of other antibody fragments. While various antibody fragments are defined in terms of the digestion of an intact antibody, one of skill will appreciate that such Fab′ fragments may be synthesized de novo either chemically or by utilizing recombinant DNA methodology. Antibodies also include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The phrases “an effective amount” and “an amount sufficient to” refer to amounts of a biologically active agent that produce an intended biological activity.

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, terms have the meanings ascribed to them unless specified otherwise. All publications, patent applications, patents and other references mentioned herein are incorporated by reference in their entirety. The materials, methods, and examples disclosed herein are illustrative only and not intended to be limiting.

It has also been determined that activating the LIGHT pathway in a cell can result in the induction of inflammation. Accordingly, reference to “modulating” inflammation of a cell “relative to” normal cell characteristics should be understood to include the over-expression of LIGHT pathway levels results in the induction of inflammation that is not generally observed in the context of cells that do not express LIGHT pathway at a functional level.

As used herein, “level” or “functional level” of LIGHT pathway activation should be understood as a reference to the level of LIGHT pathway activity which is present in any given cell. Although an increase in the concentration of LIGHT pathway proteins will generally correlate to an increase in the level of LIGHT pathway functional activity which is observed in a cell, the person skilled in the art would also understand that increases in the level of activity can be achieved by means other than merely increasing absolute intracellular LIGHT pathway concentrations. For example, one might utilize forms of LIGHT pathway which exhibit an increased half-life or otherwise exhibit enhanced activity. Reference to “over-expressing” the subject LIGHT pathway level should therefore be understood as a reference to up regulating intracellular LIGHT pathway to an effective functional level which is greater than that expressed under the normal physiological conditions for a given cell prior to inflammation or to the up-regulation of LIGHT pathway levels to any level of functionality but where that up-regulation event is one which is artificially effected rather than being an increase which has occurred in the subject cell due to the effects of naturally occurring physiology prior to inflammation. Accordingly, this latter form of up-regulation may correlate to up-regulating LIGHT pathway to levels which fall within the normal physiological range but which are higher than pre-stimulation or pre-inflammation levels. The mechanism by which up-regulation is achieved may be artificial mechanism that seek to mimic a physiological pathway—for example introducing a hormone or other stimulatory molecule, e.g., retinoic acid (RA). Accordingly, the term “expressing” is not intended to be limited to the notion of LIGHT pathway gene transcription and translation. Rather, it is a reference to an outcome, being the establishment of a higher and effective functional level of LIGHT pathway than is found under normal physiological conditions in a cell at a particular point in time (e.g., it includes non-naturally occurring increases in LIGHT pathway level, even where those increases may fall within the normal physiological range which one might observe). Reference to the subject functional level being an “effective” level should be understood as a level of over-expression which achieves the modulation of inflammation of a cell relative to a normal cell.

Reference to “modulating” in the context of cell inflammation includes, for example, inducing or reducing the inflammation, scleroderma or hepatitis In the context of the functional level of LIGHT pathway, reference to “modulating” includes, for example, up regulating or down regulating the functional level of LIGHT pathway activity. Determining the specific functional level (e.g., “effective” level) to which the LIGHT pathway activity should be up or down-regulated to achieve the desired phenotypic change for any given cell type is a matter of routine procedure. The person of skill in the art would be familiar with methods of determining such a level. “Modulating cellular inflammation,” as used herein includes, any up or down-regulation of inflammation. It also includes initiation or advancing the stage of inflammation or the alleviation of inflammation. Modulation, as used herein may also refer to clearance of bacteria, liver inflammation, infiltration of inflammatory cells, liver cirrhosis, hepatocellular carcinoma and/or hepatocyte necrosis. These are measures of a LIGHT pathway related disorder. Modulating one or more of these may indicated that a test compound may be useful in treating a LIGHT pathway related disorder.

Methods of Treating

In one aspect, provided herein are methods to treat, prevent, ameliorate, reduce or alleviate a LIGHT pathway related disorder or symptoms thereof, comprising: administering to a subject in need thereof a composition comprising a pharmaceutically effective amount of LIGHT pathway protein modulator, e.g., a composition that blocks, either directly or indirectly the association of LIGHT and LTβR.

An “effective amount” includes, for example, an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

In one embodiment, the composition is administered to the subject orally, intravenously, intrathecally or epidurally, intramuscularly, subcutaneously, perineurally, intradermally, topically or transcutaneously.

Subjects include mammals, e.g., humans, cows, pigs, horses, squirrels, primates, dogs, cats, rabbits, goats, etc.

“Obtaining the LIGHT pathway modulator,” as used herein refers to making or buying the modulator.

In one embodiment, a LIGHT pathway related disorder or symptom thereof is indicated by alleviation of pain, jaundice (yellowing of the skin and eyes); fatigue; abdominal pain; loss of appetite; nausea; vomiting; diarrhea; low grade fever, clearance of bacteria, liver inflammation, infiltration of inflammatory cells, hepatocyte necrosis, liver cirrhosis (replacement of liver cells by connective tissues), hepatocellular carcinoma and/or headache.

Reference herein to “teatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered to include reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, for example, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in herein may be co-administered with one or more other compounds or molecules. By “co-administered” is meant simultaneous or sequential administration in the same formulation or in two different formulations via the same or different routes or sequential administration by the same or different routes. For example, the subject LIGHT pathway may be administered together with an agonistic agent in order to enhance its effects. Alternatively, in the case of organ tissue transplantation, the LIGHT pathway may be administered together with immunosuppressive drugs. By “sequential” administration is meant a time difference of from seconds, minutes, hours or days between the administration of the two types of molecules. These molecules may be administered in any order. In another embodiment, the composition further comprises a therapeutically effective amount of one or more of at least one anticonvulsant, non-narcotic analgesic, non-steroidal anti-inflammatory drug, antidepressant, glutamate receptor antagonist, nicotinic receptor antagonist, or local anesthetic.

Another aspect of the present invention relates to the use of an agent capable of modulating the functional level of LIGHT pathway (e.g., level of soluble LIGHT) in the manufacture of a medicament for the modulation of cell inflammation in a mammal wherein disrupting or modulating an interaction between LIGHT and LTβR modulates inflammation of the cells.

In another aspect, the present invention relates to the use of LIGHT pathway or a nucleic acid encoding LIGHT pathway in the manufacture of a medicament for the modulation of cell inflammation in a mammal wherein modulating an interaction between LIGHT and LTβR modulates inflammation of the cells.

“Aberrant or otherwise unwanted cellular inflammation” refers, for example, to conditions in a mammal, wherein inflammation desired and not occurring or vice verse. Aberrant inflammation may happen, for example, one or more of a neuronal cell, a pancreatic cell, a lung cell, bone tissue cell, a spleen cell, heart cell, kidney cell, a testis cell, or an intestinal tract cell. The aberrant inflammation may lead, for example, to one or more of the following conditions: e.g., liver inflammation, hepatitis (autoimmune and pathogen induced), arthrosclerosis, arthritis, IgA nephropathy, inflammatory bowel disease, liver cirrhosis, and hepocellular carcinoma. The inflammation may be managed, for example, by modulating an interaction between LIGHT and LTβR.

The modulation may be the down-regulation of a LIGHT pathway protein level or fragment thereof and the down-regulation for example by the introduction a nucleic acid molecule encoding an interfering nucleic acid molecule or a protein molecule, e.g., an RNAi protein or functional equivalent. The modulation may also be by contacting the cell with a compound that modulates transcriptional and/or translational regulation of a LIGHT pathway gene. The modulation may also be by contacting the cell with a compound that functions as an agonist of the protein expression product.

In the one embodiment, the modulation is down-regulation of LIGHT pathway protein levels and the down-regulation may be done by contacting the cell with a compound that functions as an antagonist to the LIGHT pathway protein expression product.

In the one embodiment, the modulation is prevention of cleavage of LIGHT to form the soluble form of LIGHT.

In either up- or down-regulation, the modulation of inflammation may be in vivo or in vitro.

In one aspect, provided herein are uses of LIGHT pathway, or homologues, derivatives or fragments thereof, for the manufacture of a medicament to treat LIGHT pathway related disorders.

Provided herein, according to one aspect, are pharmaceutical compositions comprising a pharmaceutically effective amount of a LIGHT pathway modulator effective to treat, prevent, ameliorate, reduce or alleviate a LIGHT pathway related disorder or symptoms thereof and a pharmaceutically acceptable excipient.

In one embodiment, the LIGHT pathway modulator is selected from one or more of a small molecule, an anti-LIGHT pathway antibody, an antigen-binding fragment of an anti-LIGHT pathway antibody, a polypeptide, a peptidomimetic, a nucleic acid encoding a peptide, or an organic molecule.

The present invention provides for both prophylactic and therapeutic methods of treating a subject at risk of, or susceptible to, a LIGHT pathway related disease or disorder. Treatment is defined as the application or administration of a therapeutic agent to a patient, or application or administration of a therapeutic agent to an isolated tissue or cell line from a patient, who has a LIGHT pathway related disease or disorder, a symptom of a LIGHT pathway related disease or disorder or a predisposition toward a LIGHT pathway related disease or disorder, with the purpose to cure, heal, alleviate, relieve, alter, remedy, ameliorate, improve or affect the disease or disorder, the symptoms of the disease or disorder or the predisposition toward the disease or disorder.

The therapeutic methods of the invention involve the administration of the polypeptide and/or nucleic acid molecules of the invention as described herein.

In one aspect, the invention provides a method for preventing a LIGHT pathway related disease or disorder in a subject by administering to the subject a polypeptide or nucleic acid molecule of the invention as described herein.

The invention provides therapeutic methods and compositions for the prevention and treatment of a LIGHT pathway related disease or disorder. In particular, the invention provides methods and compositions for the prevention and treatment of the disease or disorder in subjects.

In one embodiment, the present invention contemplates a method of treatment, comprising: a) providing, i.e., administering: i) a mammalian patient particularly human who has, or is at risk of developing a LIGHT pathway disease or disorder, ii) one or more molecules of the invention as described herein.

The term “at risk for developing” is herein defined as individuals an increased probability of contracting an LIGHT pathway related disease or disorder due to exposure or other health factors. For example, contact with a hepatitis virus, e.g., hepatitis A-E.

The present invention is also not limited by the degree of benefit achieved by the administration of the molecule. For example, the present invention is not limited to circumstances where all symptoms are eliminated. In one embodiment, administering a molecule reduces the number or severity of symptoms of a LIGHT pathway related disease or disorder. In another embodiment, administering of a molecule may delay the onset of symptoms of a LIGHT pathway related disease or disorder.

Yet another aspect of this invention relates to a method of treating a subject (e.g., mammal, human, horse, dog, cat, mouse) having a disease or disease symptom (including, but not limited to e.g., liver inflammation, hepatitis (autoimmune and pathogen induced), arthrosclerosis, arthritis, IgA nephropathy, inflammatory bowel disease, liver cirrhosis, and hepocellular carcinoma. The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Typical subjects for treatment in accordance with the individuals include mammals, such as primates, preferably humans. Cells treated in accordance with the invention also preferably are mammalian, particularly primate, especially human. As discussed above, a subject or cells are suitably identified as in, needed of treatment, and the identified cells or subject are then selected for treatment and administered one or more of fusion molecules of the invention.

The treatment methods and compositions of the invention also will be useful for treatment of mammals other than humans, including for veterinary applications such as to treat horses and livestock e.g., cattle, sheep, cows, goats, swine and the like, and pets such as dogs and cats.

In other embodiments, the inhibition LIGHT pathway protein family members can be achieved by any available means, e.g., inhibition of: (1) the expression, mRNA stability, protein trafficking, modification (e.g., phosphorylation), or degradation of an protein family member, or (2) one or more of the normal functions of an protein family member.

In one embodiment, LIGHT pathway protein family member inhibition is achieved by reducing the level of LIGHT pathway protein family members in a tissue expressing the protein. Thus, the method of the invention can target protein family members in tissues wherein the protein is expressed as described infra. This can be achieved using, e.g., antisense or RNA interference (RNAi) techniques to reduce the level of the RNA available for translation.

Methods of Screening

The role of protein family members in mediating a LIGHT pathway related disorders makes the LIGHT pathway protein family member an attractive target for agents that modulate these disorders to effectively treat, prevent, ameliorate, reduce or alleviate the disorders. Accordingly, the invention provides prescreening and screening methods aimed at identifying such agents. The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof or in animals.

In one embodiment, therefore, a prescreening method comprises contacting a test agent with an LIGHT pathway protein family member or members (e.g., soluble LIGHT and LTβR). Such prescreening is generally most conveniently accomplished with a simple in vitro binding assay or in vitro disruption of biding assay. Means of assaying for specific binding of a test agent to a polypeptide are well known to those of skill in the art and are detailed in the Examples infra. In one binding assay, the polypeptide is immobilized and exposed to a test agent (which can be labeled), or alternatively, the test agent(s) are immobilized and exposed to the polypeptide (which can be labeled) or one of the proteins is immobilized and the test agent(s) and other protein are exposed to the immobilized protein. The immobilized species is then washed to remove any unbound material and the bound material is detected. To prescreen large numbers of test agents, high throughput assays are generally preferred. Various screening formats are discussed in greater detail below.

Test agents, including, for example, those identified in a prescreening assay of the invention can also be screened to determine whether the test agent affects the levels of protein family members or RNA. Agents that reduce these levels can potentially reduce one or more LIGHT pathway related disorders.

Accordingly, the invention provides a method of screening for an agent that modulates a LIGHT pathway related disorder in which a test agent is contacted with a cell that expresses a protein family member in the absence of test agent. Preferably, the method is carried out using an in vitro assay or in vivo. In such assays, the test agent can be contacted with a cell in culture or to a tissue. Alternatively, the test agent can be contacted with a cell lysate or fraction thereof (e.g., a membrane fraction for detection of protein family members or polypeptides thereof). The level of (i) protein family members; or RNA is determined in the presence and absence (or presence of a lower amount) of test agent to identify any test agents that alter the level. If the level assayed is altered, the test agent is selected as a potential modulator of a LIGHT pathway related disorder. In a preferred embodiment, an agent that reduces or increases the level assayed is selected as a potential modulator of one or more LIGHT pathway related disorders.

Cells useful in this screening method include those from any of the species described above in connection with the method of reducing a drug-related effect or behavior. Cells that naturally express an protein family member are useful in this screening methods. Examples include PC12 cells, SH-SY5y cells, NG108-15 cells, IMR-32 cells, SK-N-SH cells, RINm5F cells, and NMB cells. Alternatively, cells that have been engineered to express a protein family member can be used in the method.

Mice useful in screening include normal mice and mice having one or more of a Y173F LIGHT or a LIGHTΔL protein.

The mice may contain, for example SEQ ID NO.: 1: (Y173F LIGHT):

MESVVQPSVFVVDGQTDIPFRRLEQNHRRRRCGTVQVSLALVLLLGAGLA
TQGWFLLRLHQRLGDIVAHLPDGGKGSWEKLIQDQRSHQANPAAHLTGAN
ASLIGIGGPLLWETRLGLAFLRGLTYHDGALVTMEPGYYYVYSKVQLSGV
GCPQGLANGLPITHGLYKRTSRFPKELELLVSRRSPCGRANSSRVWWDSS
FLGGVVHLEAGEEVVVRVPGNRLVRPRDGTRSYFGAFMV

The mice may contain, for example SEQ ID NO.: 1: (LIGHTΔL):

MESVVQPSVFVVDGQTDIPFRRLEQNHRRRRCGTVQVSLALVLLLGAGLA
TQGWFLLRLHQRQRSHQANPAAHLTGANASLIGIGGPLLWETRLGLAFLR
GLTYHDGALVTMEPGYYYVYSKVQLSGVGCPQGLANGLPITHGLYKRTSR
YPKELELLVSRRSPCGRANSSRVWWDSSFLGGVVHLEAGEEVVVRVPGNR
LVRPRDGTRSYFGAFMV

According to another aspect, method for screening a therapeutic agent to treat, prevent, ameliorate, reduce or alleviate a LIGHT pathway related disorder or symptoms thereof may comprise administering a test agent to an animal, and measuring modulation of one or more of inflammation, clearance of bacteria, liver inflammation, infiltration of inflammatory cells, liver cirrhosis, and hepatocellular carcinoma and/or hepatocyte necrosis. Animals useful in the study include mice, primates, rabbits and other useful animal and cellular or organ models known to one of skill in the art. Mice expressing one or more of a Y173F LIGHT or a LIGHTΔL protein are also useful in the screening methods. It is also useful to induce hepatitis in the animal, cell or organ model, which may comprise inducement by treatment with c concanavalian A, listeria monocytogenes, hepatitis viruses, autoimmune hepatitis, acetoaminophen-induce hepatocyte death or alcohol-induce hepatitis. Test agents that are found to decrease inflammation or soluble LIGHT level indicate that the test agent may be useful in treating a LIGHT pathway disorder. Also, agents that reduce inflammation, reduce clearance of bacteria, reduce liver inflammation, reduce infiltration of inflammatory cells, liver cirrhosis, hepatocellular carcinoma and/or reduce hepatocyte necrosis may also be useful in treating a LIGHT pathway disorder, e.g., hepatitis.

In one embodiment, the test agent is contacted with the cell in the presence of a drug. The drug is generally one that produces one or more undesirable effects or behaviors, such as, for example, sedative-hypnotic and analgesic-drugs. In particular embodiments, the drug is ethanol, a cannabinioid, or an opioid.

As noted above, screening assays are generally carried out in vitro, for example, in cultured cells, in a biological sample (e.g., brain, dorsal root ganglion neurons, and sympathetic ganglion neurons), or fractions thereof. For ease of description, cell cultures, biological samples, and fractions are referred to as “samples” below. The sample is generally derived from an animal (e.g., any of the research animals mentioned above), preferably a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

protein family members can be detected and quantified by any of a number of methods well known to those of skill in the art. Examples of analytic biochemical methods suitable for detecting protein family member, include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), receptor-linked immunosorbent assays (ELISAs), immunofluorescent assays, Western blotting, fluorescence resonance energy transfer (FRET) assays, yeast two-hybrid assays, whole or partial cell current recordings, and the like. Peptide modulators may be discovered or screened for example, by phage display. See 5,096,815; 5,198,346; 5,223,409; 5,260,203; 5,403,484; 5,534,621; and 5,571,698.

Methods for identifying lead compounds for a pharmacological agent useful in the treatment of a LIGHT pathway related disorder comprising contacting a protein with a test compound, and measuring inflammation. The LIGHT pathway protein may also be a modified, e.g., a chimeric and/or a deletion mutant. The protein may be isolated or may be in a membrane or an artificial membrane. The contacting may be directly or indirectly.

Methods of the invention also include methods for screening a therapeutic agent to treat, prevent, ameliorate, reduce or alleviate a LIGHT pathway related disorder or symptoms thereof, comprising administering a test agent to a mouse having an over-expressed protein.

The proteinaceous molecules described above may be derived from any suitable source such as natural, recombinant or synthetic sources and includes fusion proteins or molecules which have been identified following, for example, natural product screening or high-throughput screening. The reference to non-proteinaceous molecules may be, for example, a reference to a nucleic acid molecule or it may be a molecule derived from natural sources, such as for example natural product screening, or may be a chemically synthesized molecule. The present invention contemplates analogues of the LIGHT pathway expression product or small molecules capable of acting as agonists or antagonists. Chemical agonists may not necessarily be derived from the LIGHT pathway expression product but may share certain conformational similarities. Alternatively, chemical agonists may be specifically designed to meet certain physiochemical properties. Antagonists may be any compound capable of blocking, inhibiting or otherwise preventing LIGHT pathway from carrying out its normal biological function, such as molecules which prevent its activation or else prevent the downstream functioning of activated LIGHT pathway. Antagonists include monoclonal antibodies and antisense nucleic acids which prevent transcription or translation of LIGHT pathway genes or mRNA in mammalian cells. Modulation of expression may also be achieved utilizing antigens, RNA, ribosomes, DNAzymes, RNA aptamers, antibodies or molecules suitable for use in co-suppression. The proteinaceous and non-proteinaceous molecules referred to in points (i)-(v), above, are herein collectively referred to as “modulatory agents”. In another embodiment, the modulator is one or more of a small molecule, an anti-LIGHT pathway antibody, an antigen-binding fragment of an anti-LIGHT pathway antibody, a polypeptide, a peptidomimetic, a nucleic acid encoding a peptide, or an organic molecule.

Screening for the modulatory agents can be achieved by any one of several suitable methods including, but in no way limited to, contacting a cell comprising one or more LIGHT pathway gene or functional equivalent or derivative thereof with an agent and screening for the modulation of soluble LIGHT protein production or functional activity, modulation of the expression of a nucleic acid molecule encoding LIGHT pathway or modulation of the activity or expression of a downstream LIGHT pathway cellular target, e.g., soluble LIGHT. Detecting such modulation can be achieved utilizing techniques such as Western blotting, electrophoretic mobility shift assays and/or the readout of reporters of LIGHT pathway activity such as luciferases, CAT and the like or observation of morphological changes.

The LIGHT pathway gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate LIGHT pathway activity, at either the nucleic acid or expression product levels, or the gene may require activation—thereby providing a model useful for, inter alia, screening for agents which up regulate LIGHT pathway expression. Further, to the extent that a LIGHT pathway nucleic acid molecule is transfected into a cell, that molecule may comprise the entire LIGHT pathway gene or it may merely comprise a portion of the gene such as the portion which regulates expression of the LIGHT pathway product. For example, the LIGHT pathway promoter region may be transfected into the cell which is the subject of testing. In this regard, where only the promoter is utilized, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream LIGHT pathway regulatory target, rather than LIGHT pathway itself. Yet another example includes LIGHT pathway binding sites ligated to a minimal reporter. For example, modulation of LIGHT pathway activity can be detected by screening for the modulation of the functional activity in a cell. This is an example of an indirect system where modulation of LIGHT pathway expression, per se, is not the subject of detection. Rather, modulation of the molecules which LIGHT pathway regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the LIGHT pathway nucleic acid molecule or expression product itself or which modulate the expression of an upstream molecule, which upstream molecule subsequently modulates LIGHT pathway expression or expression product activity. Accordingly, these methods provide a mechanism for detecting agents which either directly or indirectly modulate LIGHT pathway expression and/or activity.

The agents which are utilized in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, The agent is associated with a molecule which permits its targeting to a localized region.

The proteinaceous or non-proteinaceous molecules may act either directly or indirectly to modulate the expression of LIGHT pathway or the activity of the LIGHT pathway expression product. The molecule acts directly if it associates with the LIGHT pathway nucleic acid molecule or expression product to modulate expression or activity, respectively. The molecule acts indirectly if it associates with a molecule other than the LIGHT pathway nucleic acid molecule or expression product which other molecule either directly or indirectly modulates the expression or activity of the LIGHT pathway nucleic acid molecule or expression product, respectively. Accordingly, the method of the present invention encompasses the regulation of LIGHT pathway nucleic acid molecule expression or expression product activity via the induction of a cascade of regulatory steps.

The term “expression” refers, for example, to the transcription and translation of a nucleic acid molecule. Reference to “expression product” is a reference to the product produced from the transcription and translation of a nucleic acid molecule.

“Derivatives” of the molecules herein described (for example LIGHT pathway or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, LIGHT pathway or derivative thereof may be fused to a molecule to facilitate its entry into a cell. Analogues of the molecules contemplated herein include, for example, modification to side chains, incorporating of unnatural amino acids and/or their derivatives during peptide, polypeptide or protein synthesis and the use of crosslinkers and other methods including conformational constraints on the proteinaceous molecules or their analogues.

Derivatives of nucleic acid sequences which may be utilized in accordance with the method described herein may similarly be derived from single or multiple nucleotide substitutions, deletions and/or additions including fusion with other nucleic acid molecules. The derivatives of the nucleic acid molecules utilized as described herein include, for example, oligonucleotides, PCR primers, antisense molecules, molecules suitable for use in co-suppression and fusion of nucleic acid molecules. Derivatives of nucleic acid sequences also include degenerate variants.

A “variant” of LIGHT pathway should be understood to include, for example, molecules that exhibit at least some of the functional activity of the form of LIGHT pathway of which it is a variant. A variation may take any form and may be naturally or non-naturally occurring. A mutant molecule is one which exhibits, for example, modified functional activity.

A “homologue” is includes, for example, that the molecule is derived from a species other than that which is being treated in accordance with the method of the present invention. This may occur, for example, where it is determined that a species other than that which is being treated produces a form of LIGHT pathway which exhibits similar and suitable inflammation to that of the LIGHT pathway which is naturally produced by the subject undergoing treatment.

Chemical and functional equivalents include, for example, molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

In one aspect, provided herein are methods for screening a therapeutic agent to treat, prevent, ameliorate, reduce or alleviate a LIGHT pathway related disorder or symptoms thereof, comprising administering a test agent to a mouse having an over-expressed protein, and measuring modulation of inflammation. In one aspect, provided herein are methods for identifying lead compounds for a pharmacological agent useful in the treatment of a LIGHT pathway related disorder comprising contacting a cell expressing a protein with a test compound, and measuring LIGHT pathway expression, modulation, or inflammation or modulation of GDPD activity (e.g., glycerophosphodiesterase activity).

In one aspect, provided herein are methods for identifying lead compounds for a pharmacological agent useful in the treatment of a LIGHT pathway related disorder comprising contacting a cell that does not express a functional amount of a protein with a test compound, and measuring one or more of LIGHT pathway expression or inflammation.

In one embodiment, LIGHT pathway expression or inflammation is measured by one or more of measuring protein or RNA expression, observing physical inflammation markers, measuring protein or RNA levels of one or more of LTβR or LIGHT.

In another embodiment, the test compounds is one or more of a peptide, a small molecule, an antibody or fragment thereof, and nucleic acid or a library thereof.

Also useful in the screening techniques described herein are combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing LIGHT pathway analogues which exhibit properties such as more potent pharmacological effects.

With respect to high throughput library screening methods, oligomeric or small-molecule library compounds capable of interacting specifically with a selected biological agent, such as a biomolecule, a macromolecule complex, or cell, are screened utilizing a combinational library device which is easily chosen by the person of skill in the art from the range of well-known methods, such as those described above. In such a method, each member of the library is screened for its ability to interact specifically with the selected agent. In practicing the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of LIGHT pathway, it may also be desirable to identify and utilize molecules which function agonistically or antagonistically to LIGHT pathway in order to up or down-regulate the functional activity of LIGHT pathway in relation to modulating cell inflammation. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of LIGHT pathway capable of acting as agonists or antagonists. Chemical agonists may not necessarily be derived from LIGHT pathway but may share certain conformational similarities. Alternatively, chemical agonists may be specifically designed to mimic certain physiochemical properties of LIGHT pathway. Antagonists may be any compound capable of blocking, inhibiting or otherwise preventing LIGHT pathway from carrying out its normal biological functions. Antagonists include monoclonal antibodies specific for LIGHT pathway or parts of LIGHT pathway.

Analogues of LIGHT pathway or of LIGHT pathway agonistic or antagonistic agents contemplated herein include, for example, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

High Throughput Screening Assays

High throughput screening (HTS) typically uses automated assays to search through large numbers of compounds for a desired activity. Typically HTS assays are used to find new drugs by screening for chemicals that act on a particular receptor or molecule. For example, if a chemical inactivates an receptor it might prove to be effective in preventing a process in a cell which causes a disease. High throughput methods enable researchers to try out thousands of different chemicals against each target very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day. Examples include the yeast two-hybrid system and phage display. For examples of phage display see, U.S. Pat. Nos. 5,096,815; 5,198,346; 5,223,409; 5,260,203; 5,403,484; 5,534,621; and 5,571,698.

To achieve high-throughput screening, it is best to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known modulator (or activator) of an receptor for which modulators are sought, can be incubated with one sample of the assay, and the resulting decrease (or increase) in the receptor activity determined according to the methods herein. It will be appreciated that modulators can also be combined with the receptor activators or modulators to find modulators which inhibit the receptor activation or repression that is otherwise caused by the presence of the known the receptor modulator. Similarly, when ligands to a sphingolipid target are sought, known ligands of the target can bc present in control/calibration assay wells.

Measuring Binding Reactions During Screening Assays

Techniques for measuring the progression of binding reactions in multicontainer carriers are known in the art and include, but are not limited to, the following.

Spectrophotometric and spectrofluorometric assays are well known in the art. Examples of such assays include the use of colorimetric assays for the detection of peroxides, as disclosed in Example 1(b) and Gordon, A. J. and Ford, R. A., The Chemist's Companion: A Handbook Of Practical Data, Techniques, And References, John Wiley and Sons, N.Y., 1972, Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., Spectrophotometry and Spectrofluorometry: A Practical Approach, pp. 91-114, IRL Press Ltd. (1987); and Bell, Spectroscopy In Biochemistry, Vol., pp. 155-194, CRC Press (1981).

In spectrofluorometric methods, receptors are exposed to substrates that change their intrinsic fluorescence when processed by the target receptor. Typically, the substrate is nonfluorescent and converted to a fluorophore through one or more reactions. As a non-limiting example, SMase activity can be detected using the Amplex®. Red reagent (Molecular Probes, Eugene, Oreg.). In order to measure sphingomyelinase activity using Amplex Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (e.g., a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., Application of Fluorescence Polarization Assays in High-Throughput Screening, Genetic Engineering News, 17:27, 1997.

FP is particularly desirable since its readout is independent of the emission intensity (Checovich, W. J., et al., Nature 375:254-256, 1995; Dandliker, W. B., et al., Methods in Enzymology 74:3-28, 1981) and is thus insensitive to the presence of colored compounds that quench fluorescence emission. Fluorescence Polarization (FP) and FRET (see below) are well-suited for identifying compounds that block interactions between sphingolipid receptors and their ligands. See, for example, Parker et al., Development of high throughput screening assays using fluorescence polarization: nuclear receptor-ligand-binding and kinase/phosphatase assays, J Biomol Screen 5:77-88, 2000.

Fluorophores derived from sphingolipids that may be used in FP assays are commercially available. For example, Molecular Probes (Eugune, Oreg.) currently sells sphingomyelin and one ceramide fluorophores. These are, respectively, N-(4,4-difluoro-5,7-dimethyl-4-bora-3a,4a-diaza-s-inda-cene-3-pentanoyl)sphingosyl phosphocholine (BODIPY®. FL C5-sphingomyelin); N-4,4-difluoro-5,7-dimethyl-4-bora-3a,4a-diaza-s-inda-cene-3-dodecanoyl)sphingosyl phosphocholine (BODIPY®. FL C12-sphingomyelin); and N-(4,4-difluoro-5,7-dimethyl-4-bora-3a,4a-diaza-s-indacene-3-pentanoyl)sphingosine (BODIPY®. FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION fluorescence polarization system (Tecan A G, Hombrechtikon, Switzerland). General multiwell plate readers for other assays are available, such as the VERSAMAX reader and the SPECTRAMAX multiwell plate spectrophotometer (both from Molecular Devices).

Fluorescence resonance energy transfer (FRET) is another useful assay for detecting interaction and has been described previously. See, e.g., Heim et al., Curr. Biol. 6:178-182, 1-996; Mitra et al., Gene 173:13-17 1996; and Selvin et al., Meth. Enzymol. 246:300-345, 1995. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., J. Lipid Res. 38:2365-2373 (1997); Kahl et al., Anal. Biochem. 243:282-283 (1996); Undenfriend et al., Anal. Biochem. 161:494-500 (1987)). See also U.S. Pat. Nos. 4,626,513 and 4,568,649, and European Patent No. 0,154,734. One commercially available system uses FLASHPLATE scintillant-coated plates (NEN Life Science Products, Boston, Mass.).

The target molecule can be bound to the scintillator plates by a variety of well known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells.

The assay can be measured immediately or allowed to reach equilibrium. Either way, when a radiolabel becomes sufficiently close to the scintillant coating, it produces a signal detectable by a device such as a TOPCOUNT NXT microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., Anal. Biochem. 257:112-119, 1998).

In one embodiment, protein family members are detected/quantified using a ligand binding assay, such as, for example, a radioligand binding assay. Briefly, a sample from a tissue expressing protein family members is incubated with a suitable ligand under conditions designed to provide a saturating concentration of ligand over the incubation period. After ligand treatment, the sample is assayed for radioligand binding. Any ligand that binds to protein family members can be employed in the assay. Any of the protein family member modulators discussed above can, for example, be labeled and used in this assay. An exemplary, preferred ligand for this purpose is ¹²⁵I-omega-conotoxin GVIA. Binding of this ligand to cells can be assayed as described, for example, in Solem et al. (1997) J. Pharmacol. Exp. Ther. 282:1487-95. Binding to membranes (e.g., brain membranes) can be assayed according to the method of Wagner et al. (1995) J. Neurosci. 8:3354-3359 (see also, the modifications of this method described in McMahon et al. (2000) Mol. Pharm. 57:53-58).

Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence LIGHT pathway polypeptide(s) in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In certain embodiments, LIGHT pathway polypeptide(s) are detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., 3H, 125I, 35S, 14C, or 32P), receptors (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In preferred embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Changes in protein family member subunit expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In one embodiment, amplification-based assays can be used to detect, and optionally quantify, a polynucleotide encoding a protein of interest. In such amplification-based assays, the mRNA in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of the LIGHT pathway mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of LIGHT pathway mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the LIGHT pathway mRNA of interest, and standard methods (such as, e.g., densitometry where the nucleic acid probe is radioactively labeled) can be used to quantify the level of the LIGHT pathway polynucleotide.). A variety of additional nucleic acid hybridization formats are known to those skilled in the art. Standard formats include sandwich assays and competition or displacement assays. Sandwich assays are commercially useful hybridization assays for detecting or isolating polynucleotides.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). See also, for example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

Hybridization assays according to the invention can also be carried out using a MicroElectroMechanical System (MEMS), such as the Protiveris' multicantilever array.

LIGHT pathway RNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

In a preferred embodiment, generally involving the screening of a large number of test agents, the screening method includes the recordation of any test agent selected in any of the above-described prescreening or screening methods in a database of agents that may modulate inflammation. The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

Test Agents Identified by Screening

When a test agent is found to modulate one or more LIGHT pathway protein family members, or RNA. A preferred screening method of the invention further includes combining the test agent with a carrier, preferably pharmaceutically acceptable carrier, such as are described above. Generally, the concentration of test agent is sufficient to alter the level of protein family members or RNA, or inflammation. This concentration will vary, depending on the particular test agent and specific application for which the composition is intended. As one skilled in the art appreciates, the considerations affecting the formulation of a test agent with a carrier are generally the same as described above with respect to methods of reducing a drug-related effect or behavior.

In a preferred embodiment, the test agent is administered to an animal to measure the ability of the selected test agent to modulate a drug-related effect or behavior in a subject, as described in greater detail below.

Preferred compositions for use in the therapeutic methods of the invention inhibit the protein family member function by about 5% based on, for example, compound state analysis techniques or modulatory profiles described infra, more preferably about 7.5% or 10% inhibition or initiation of inflammation of the cell, and still more preferable, at least about 20%, 30%, 40%, 50%, 60%, 70%, 80%, 90% or 100% initiation or inhibition of inflammation.

Compositions

Soluble polypeptides derived from LIGHT pathway protein family member, or other proteins that have the ability to block the interaction between LIGHT and LTβR are useful. In addition, modification of such residues may permit the skilled artisan to tailor the binding specificities and/or affinity of polypeptides.

The LIGHT pathway protein family members are of particular interest because they are of interest in the treatment, prevention, amelioration, reduction or alleviation of diseases.

The polypeptides may be prepared in various ways including, for example, molecular biological techniques, including proteolytic digestion of cells or cellular membrane preparations comprising the receptor (Bartfeld et al., Active acetylcholine receptor fragment obtained by tryptic digestion of acetylcholine receptor from Torpedo californica, Biochem Biophys Res Commun. 89:512-9, 1979; Borhani et al., Crystallization and X-ray diffraction studies of a soluble form of the human transferrin receptor, J Mol. Biol. 218:685-9, 1991), recombinant DNA technologies (Marlovits et al., Recombinant soluble low-density lipoprotein receptor fragment inhibits common cold infection, J Mol Recognit. 11:49-51, 1998; Huang et al., Expression of a human thyrotrophin receptor fragment in Escherichia coli and its interaction with the hormone and autoantibodies from subjects with Graves' disease, J Mol Endocrinol. 8:137-44, 1992), or by in vitro synthesis of oligopeptides.

Peptidomimetics

In general, a polypeptide mimetic (“peptidomimetic”) is a molecule that mimics the biological activity of a polypeptide, but that is not peptidic in chemical nature. While, in certain embodiments, a peptidomimetic is a molecule that contains no peptide bonds (that is, amide bonds between amino acids), the term peptidomimetic may include molecules that are not completely peptidic in character, such as pseudo-peptides, semi-peptides and peptoids. Examples of some peptidomimetics by the broader definition (e.g., where part of a polypeptide is replaced by a structure lacking peptide bonds) are described below. Whether completely or partially non-peptide in character, peptidomimetics according to this invention may provide a spatial arrangement of reactive chemical moieties that closely resembles the three-dimensional arrangement of active groups in a polypeptide. As a result of this similar active-site geometry, the peptidomimetic may exhibit biological effects that are similar to the biological activity of a polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that may be obviated with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure, shape or reactivity. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean (1994), BioEssays, 16: 683-687; Cohen and Shatzmiller (1993), J. Mol. Graph., 11: 166-173; Wiley and Rich (1993), Med. Res. Rev., 13: 327-384; Moore (1994), Trends Pharmacol. Sci., 15: 124-129; Hruby (1993), Biopolymers, 33: 1073-1082; Bugg et al. (1993), Sci. Am., 269: 92-98, all incorporated herein by reference].

Specific examples of peptidomimetics are set forth below. These examples are illustrative and not limiting in terms of the other or additional modifications.

Peptides with a Reduced Isostere Pseudopeptide Bond

Proteases act on peptide bonds. Substitution of peptide bonds by pseudopeptide bonds may confer resistance to proteolysis or otherwise make a compound less labile. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., (1993), Int. J. Polypeptide Protein Res. 41:181-184, incorporated herein by reference). Thus, the amino acid sequences of these compounds may be identical to the sequences of their parent L-amino acid polypeptides, except that one or more of the peptide bonds are replaced by an isostere pseudopeptide bond. Preferably the most N-terminal peptide bond is substituted, since such a substitution would confer resistance to proteolysis by exopeptidases acting on the N-terminus.

Peptides with a Retro-Inverso Pseudopeptide Bond

To confer resistance to proteolysis, peptide bonds may also be substituted by retro-inverso pseudopeptide bonds (Dalpozzo, et al. (1993), Int. J. Polypeptide Protein Res. 41:561-566, incorporated herein by reference). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid Derivatives

Peptoid derivatives of polypeptides represent another form of modified polypeptides that retain the structural determinants for biological activity, yet eliminate the peptide bonds, thereby conferring resistance to proteolysis (Simon, et al., 1992, Proc. Natl. Acad. Sci. USA, 89:9367-9371 and incorporated herein by reference). Peptoids are oligomers of N-substituted glycines. A number of N-alkyl groups have been described, each corresponding to the side chain of a natural amino acid.

Polypeptides

The polypeptides of this invention, including the analogs and other modified variants, may generally be prepared following known techniques. Preferably, synthetic production of the polypeptide of the invention may be according to the solid phase synthetic method. For example, the solid phase synthesis is well understood and is a common method for preparation of polypeptides, as are a variety of modifications of that technique [Merrifield (1964), J. Am. Chem. Soc., 85: 2149; Stewart and Young (1984), Solid Phase polypeptide Synthesis, Pierce Chemical Company, Rockford, Ill.; Bodansky and Bodanszky (1984), The Practice of polypeptide Synthesis, Springer-Verlag, New York; Atherton and Sheppard (1989), Solid Phase polypeptide Synthesis: A Practical Approach, IRL Press, New York].

Alternatively, polypeptides of this invention may be prepared in recombinant systems using polynucleotide sequences encoding the polypeptides. For example, fusion proteins are typically prepared using recombinant DNA technology.

Polypeptide Derivatives

A “derivative” of a polypeptide is a compound that is not, by definition, a polypeptide, i.e., it contains at least one chemical linkage that is not a peptide bond. Thus, polypeptide derivatives include without limitation proteins that naturally undergo post-translational modifications such as, e.g., glycosylation. It is understood that a polypeptide of the invention may contain more than one of the following modifications within the same polypeptide. Preferred polypeptide derivatives retain a desirable attribute, which may be biological activity; more preferably, a polypeptide derivative is enhanced with regard to one or more desirable attributes, or has one or more desirable attributes not found in the parent polypeptide.

Mutant Polypeptides: A polypeptide having an amino acid sequence identical to that found in a protein prepared from a natural source is a “wildtype” polypeptide. Mutant oligopeptides can be prepared by chemical synthesis, including without limitation combinatorial synthesis.

Mutant polypeptides larger than oligopeptides can be prepared using recombinant DNA technology by altering the nucleotide sequence of a nucleic acid encoding a polypeptide. Although some alterations in the nucleotide sequence will not alter the amino acid sequence of the polypeptide encoded thereby (“silent” mutations), many will result in a polypeptide having an altered amino acid sequence that is altered relative to the parent sequence. Such altered amino acid sequences may comprise substitutions, deletions and additions of amino acids, with the proviso that such amino acids are naturally occurring amino acids.

Thus, subjecting a nucleic acid that encodes a polypeptide to mutagenesis is one technique that can be used to prepare mutant polypeptides, particularly ones having substitutions of amino acids but no deletions or insertions thereof. A variety of mutagenic techniques are known that can be used in vitro or in vivo including without limitation chemical mutagenesis and PCR-mediated mutagenesis. Such mutagenesis may be randomly targeted (i.e., mutations may occur anywhere within the nucleic acid) or directed to a section of the nucleic acid that encodes a stretch of amino acids of particular interest. Using such techniques, it is possible to prepare randomized, combinatorial or focused compound libraries, pools and mixtures.

Polypeptides having deletions or insertions of naturally occurring amino acids may be synthetic oligopeptides that result from the chemical synthesis of amino acid sequences that are based on the amino acid sequence of a parent polypeptide but which have one or more amino acids inserted or deleted relative to the sequence of the parent polypeptide. Insertions and deletions of amino acid residues in polypeptides having longer amino acid sequences may be prepared by directed mutagenesis.

Chemically Modified Polypeptides: As contemplated by this invention, the term “polypeptide” includes those having one or more chemical modification relative to another polypeptide, i.e., chemically modified polypeptides. The polypeptide from which a chemically modified polypeptide is derived may be a wildtype protein, a mutant protein or a mutant polypeptide, or polypeptide fragments thereof, an antibody or other polypeptide ligand according to the invention including without limitation single-chain antibodies, bacterial proteins and polypeptide derivatives thereof, or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

Polypeptides with N-Terminal or C-Terminal Chemical Groups: An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al. (1993), Pharma. Res. 10: 1268-1273). Other chemical modifications which enhance serum stability include, but are not limited to, the addition of an N-terminal allyl group, consisting of a lower alkyl of from 1 to 20 carbons, such as an acetyl group, and/or the addition of a C-terminal amide or substituted amide group.

Polypeptides with a Terminal D-Amino Acid: The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

Polypeptides With Substitution of Natural Amino Acids By Unnatural Amino Acids: Substitution of unnatural amino acids for natural amino acids in a subsequence of a polypeptide can confer or enhance desirable attributes including biological activity. Such a substitution can, for example, confer resistance to proteolysis by exopeptidases acting on the N-terminus. The synthesis of polypeptides with unnatural amino acids is routine and known in the art (see, for example, Coller, et al. (1993), cited above).

Post-Translational Chemical Modifications: Different host cells will contain different post-translational modification mechanisms that may provide particular types of post-translational modification of a fusion protein if the amino acid sequences required for such modifications is present in the fusion protein. A large number (.about.100) of post-translational modifications have been described, a few of which are discussed herein. One skilled in the art will be able to choose appropriate host cells, and design chimeric genes that encode protein members comprising the amino acid sequence needed for a particular type of modification.

Glycosylation is one type of post-translational chemical modification that occurs in many eukaryotic systems, and may influence the activity, stability, pharmacogenetics, immunogenicity and/or antigenicity of proteins. However, specific amino acids must be present at such sites to recruit the appropriate glycosylation machinery, and not all host cells have the appropriate molecular machinery. Saccharomyces cerevisieae and Pichia pastoris provide for the production of glycosylated proteins, as do expression systems that utilize insect cells, although the pattern of glyscoylation may vary depending on which host cells are used to produce the fusion protein.

Another type of post-translation modification is the phosphorylation of a free hydroxyl group of the side chain of one or more Ser, Thr or Tyr residues. Protein kinases catalyze such reactions. Phosphorylation is often reversible due to the action of a protein phosphatase, an receptor that catalyzes the dephosphorylation of amino acid residues.

Differences in the chemical structure of amino terminal residues result from different host cells, each of which may have a different chemical version of the methionine residue encoded by a start codon, and these will result in amino termini with different chemical modifications.

For example, many or most bacterial proteins are synthesized with an amino terminal amino acid that is a modified form of methionine, i.e., N-formyl-methionine (fMct). Although the statement is often made that all bacterial proteins are synthesized with an fMet initiator amino acid; although this may be true for E. coli, recent studies have shown that it is not true in the case of other bacteria such as Pseudomonas aeruginosa (Newton et al., J. Biol. Chem. 274:22143-22146, 1999). In any event, in E. coli, the formyl group of fMet is usually enzymatically removed after translation to yield an amino terminal methionine residue, although the entire fMet residue is sometimes removed (see Hershey, Chapter 40, “Protein Synthesis” in: Escherichia Coli and Salmonella Typhimurium: Cellular and Molecular Biology, Neidhardt, Frederick C., Editor in Chief, American Society for Microbiology, Washington, D.C., 1987, Volume 1, pages 613-647, and references cited therein.) E. coli mutants that lack the receptors (such as, e.g., formylase) that catalyze such post-translational modifications will produce proteins having an amino terminal fMet residue (Guillon et al., J. Bacteriol. 174:4294-4301, 1992).

In eukaryotes, acetylation of the initiator methionine residue, or the penultimate residue if the initiator methionine has been removed, typically occurs co- or post-translationally. The acetylation reactions are catalyzed by N-terminal acetyltransferases (NATs, a.k.a. N-alpha-acetyltransferases), whereas removal of the initiator methionine residue is catalyzed by methionine aminopeptidases (for reviews, see Bradshaw et al., Trends Biochem. Sci. 23:263-267, 1998; and Driessen et al., CRC Crit. Rev. Biochem. 18:281-325, 1985). Amino terminally acetylated proteins are said to be “N-acetylated,” “N alpha acetylated” or simply “acetylated.”

Another post-translational process that occurs in eukaryotes is the alpha-amidation of the carboxy terminus. For reviews, see Eipper et al. Annu. Rev. Physiol. 50:333-344, 1988, and Bradbury et al. Lung Cancer 14:239-251, 1996. About 50% of known endocrine and neuroendocrine peptide hormones are alpha-amidated (Treston et al., Cell Growth Differ. 4:911-920, 1993). In most cases, carboxy alpha-amidation is required to activate these peptide hormones.

Substitutions encompass amino acid alterations in which an amino acid is replaced with a different naturally-occurring or a non-conventional amino acid residue. Such substitutions may be classified as “conservative”, in which case an amino acid residue contained in a peptide is replaced with another naturally-occurring amino acid of similar character, for example Gly to Ala, Asp to Glu, Asn to Gln or Trp to Tyr. Possible alternative amino acids include serine or threonine, aspartate or glutamate or carboxyglutamate, pro line or hydroxyproline, arginine or lysine, asparagine or histidine, histidine or asparagine, tyrosine or phenylalanine or tryptophan, aspartate or glutamate, isoleucine or leucine or valine.

It is to be understood that some non-conventional amino acids may also be suitable replacements for the naturally occurring amino acids. Substitutions encompassed by the present invention may also be “non-conservative”, in which an amino acid residue which is present in a polypeptide is substituted with an amino acid having different properties, such as naturally-occurring amino acid from a different group (e.g. substituting a charged or hydrophilic or hydrophobic amino acid with alanine), or alternatively, in which a naturally-occurring amino acid is substituted with a non-conventional amino acid. Additions encompass the addition of one or more naturally occurring or non-conventional amino acid residues. Deletions encompass the deletion of one or more amino acid residues.

One of skill in the art can identify other peptides and understands that homologues and orthologues of these molecules are useful in the compositions and methods of the instant invention. Moreover, variants of the peptides, are useful in the methods and compositions of the invention.

One of skill in the art will understand that molecules that share one or more functional activities with the molecules identified above, but have differences in amino acid or nucleic acid sequence would be useful in the compositions and methods of the invention. For example, in a preferred embodiment, a polypeptide or biologically active fragment thereof has at least about 60%, 70%, 80%, 90%, 95%, 96%, 97%, 98%, 99%, or 100% identity with the polypeptide set forth as SEQ ID NO:1-2, or a fragment or variant thereof.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48:444-453) algorithm which has been incorporated into the GAP program in the GCG software package (available at http://www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences that one of skill in the art could use to make the molecules of the invention. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to 13245 nucleic acid molecules of the invention. BLAST protein searches can be performed with the XBLAST program, score=50, wordlength=3 to obtain amino acid sequences homologous to 13245 protein molecules of the invention. To obtain gapped alignments for comparison purposes, gapped BLAST can be utilized as described in Altschul et al. (1997, Nucl. Acids Res. 25:3389-3402). When using BLAST and gapped BLAST programs, the default parameters of the respective programs (e.g., XBLAST and NBLAST) can be used.

Vectors

Another aspect of the invention pertains to vectors, preferably expression vectors, containing a nucleic acid molecule encoding the fusion molecules, or components thereof, of the invention as described above. As used herein, the term “vector” refers to a nucleic acid molecule capable of transporting another nucleic acid molecule to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid molecule of the invention in a form suitable for expression of the nucleic acid molecule in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., fusion molecules comprising a chemokine receptor ligand and a toxin moiety).

The recombinant expression vectors of the invention can be designed for expression of the polypeptides of the invention in prokaryotic or eukaryotic cells. For example, the polypeptides can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

Another aspect of the invention pertains to host cells into which a nucleic acid molecule encoding a fusion polypeptide of the invention is introduced within a recombinant expression vector or a nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a fusion polypeptide of the invention can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including phosphate or chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

Methods of Making the Molecules of the Invention

As described above, molecules of the invention may be made recombinantly using the nucleic acid molecules, vectors, host cells and recombinant organisms described above.

Alternatively, the peptide can be made synthetically, or isolated from a natural source and linked to the carbohydrate recognition domain using methods and techniques well known to one of skill in the art.

Further, to increase the stability or half life of the fusion molecules of the invention, the polypeptides may be made, e.g., synthetically or recombinantly, to include one or more peptide analogs or mimetics. Exemplary peptides can be synthesized to include D-isomers of the naturally occurring amino acid residues or amino acid analogs to increase the half life of the molecule when administered to a subject.

Pharmaceutical Compositions

The nucleic acid and polypeptide fusion molecules (also referred to herein as “active compounds”) of the invention can be incorporated into pharmaceutical compositions. Such compositions typically include the nucleic acid molecule or protein, and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions of the instant invention may also include one or more other active compounds. Alternatively, the pharmaceutical compositions of the invention may be administered with one or more other active compounds. Other active compounds that can be administered with the pharmaceutical compounds of the invention, or formulated into the pharmaceutical compositions of the invention, include, for example, anti-inflammatory compounds.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Preferred pharmaceutical compositions of the invention are those that allow for local delivery of the active ingredient, e.g., delivery directly to the location of a tumor. Although systemic administration is useful in certain embodiments, local administration is preferred in most embodiments.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, kit or dispenser together with instructions, e.g., written instructions, for administration, particularly such instructions for use of the active agent to treat against a disorder or disease as disclosed herein, including a LIGHT pathway related disorder. The container, pack, kit or dispenser may also contain, for example, a nucleic acid sequence encoding a peptide, or a peptide expressing cell.

For research and therapeutic applications, an protein family member modulator is generally formulated to deliver modulator to a target site in an amount sufficient to inhibit protein family members at that site.

Modulator compositions or peptides of the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetraacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Certain embodiments include sustained-release pharmaceutical compositions. An exemplary sustained-release composition has a semipermeable matrix of a solid hydrophobic polymer to which the modulator is attached or in which the modulator is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are in the form of shaped articles, such as films, or microcapsules.

Where the modulator is a polypeptide, exemplary sustained release compositions include the polypeptide attached, typically via epsilon-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” variant retains the desired function(s).

In another embodiment, a sustained-release composition includes a liposomally entrapped modulator. Liposomes are small vesicles composed of various types of lipids, phospholipids, and/or surfactants. These components are typically arranged in a bilayer formation, similar to the lipid arrangement of biological membranes. Liposomes containing protein family member modulators are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions can also include an modulator adsorbed onto a membrane, such as a silastic membrane, which can be implanted, as described in International Publication No. WO 91/04014.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide encoding a polypeptide modulator of protein family members. Such compositions optionally include other components, as for example, a storage solution, such as a suitable buffer, e.g., a physiological buffer. In a preferred embodiment, the composition is a pharmaceutical composition and the other component is a pharmaceutically acceptable carrier as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)pro-pyl]-n,n,n-trimethylammonium chloride (DOTMA), dioleoyl phosphotidylethanolamine (DOPE), and/or dioleoyl phosphatidylcholine (DOPC). Polynucleotides can also be entrapped in liposomes, as described above.

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

Transgenic Animals

The transgenic non-human animal may be a primate, mouse, dog, cat, sheep, horse, rabbit or other non-human animal. Cells may be isolated and cultured from the transgenic non-human animals. The cells may be used in, for example, primary cultures or established cultures. In one aspect, provided herein are uses of a transgenic animal as described herein to test therapeutic agents.

In another embodiment, a decrease inflammation indicates that the test agent may be useful in treating a LIGHT pathway disorder or changes in GDPD enzymatic activity.

Transgenic animals of the invention include animals expressing a LIGHT Phe for Tyr173 (Y173F) mutation. Another transgenic animal of the invention include one expressing a LIGHTL mutant in which the amino acids from Leu63 to Asp84 of LIGHT were removed.

The use of a transgenic animals to test therapeutic agents comprises administering the therapeutic agent to the animal and determining or measuring modulation of one or more of inflammation, clearance of bacteria, liver inflammation, infiltration of inflammatory cells, liver cirrhosis and/or hepatocellular carcinoma and/or hepatocyte necrosis.

Embodiments of the invention include the use of the ES cell lines derived from the transgenic zygote, embryo, blastocyst or non-human animal to treat human and non-human animal diseases.

The methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; and Pinkert, C A, Ed., Transgenic Animal Technology: A Laboratory Handbook, Academic Press, 1994. In certain embodiments, transgenic mice will be produced as described in Thomas et al. (1999) Immunol., 163:978-84; Kanakaraj et al. (1998) J. Exp. Med., 187:2073-9; or Yeh et al. (1997) Immunity 7:715-725.

Methods of producing the transgenic animals are well-known in the art. See for example, Hooper, M L, Embryonal Stem Cells: Introducing Planned Changes into the Animal Germline (Modeem Genetics, v. 1), Int'. Pub. Distrib., Inc., 1993; Bradley et al. (1984) Nature, 309, 255-258; Jaenisch (1988) Science, 240:1468-1474; Wilmut et al. (1997) Nature, 385: 810-813; DeBoer et al., WO 91/08216; Wang, et al. Molecular Reproduction and Development (2002) 63:437-443); Page, et al. Transgenic Res (1995) 4(6):353-360; Lebkowski, et al. Mol Cell Biol (1988) 8(10):3988-3996; “Molecular Cloning: A Laboratory Manual. Second Edition” by Sambrook, et al. Cold Spring Harbor Laboratory: 1989; “Transgenic Animal Technology: A Laboratory Handbook,” C. A. Pinkert, editor, Academic Press, 2002, 2nd edition, 618 pp.; “Mouse Genetics and Transgenics: A Practical Approach,” I. J. Jackson and C. M. Abbott, editors, Oxford University Press, 2000, 299 pp.; “Transgenesis Techniques: Principles and Protocols,” A. R. Clarke, editor, Humana Press, 2001, 351 pp.; Velander et al., Proc. Natl. Acad. Sci. USA 89:12003-12007, 1992; Hammer et al., Nature 315:680-683, 1985; Gordon et al., Science 214:1244-1246, 1981; and Hogan et al., Manipulating the Mouse Embryo: A Laboratory Manual (Cold Spring Harbor Laboratory, 2002), which are each incorporated herein by reference in their entirety.

Cells obtained from the transgenic non-human animals described herein may be obtained by taking a sample of a tissue of the animal. The cells may then be cultured. The cells preferably lack production of functional protein encoded by the nucleotide sequence comprising SEQ ID NO: 1-3 or a fragments or variants thereof.

In one embodiment, the transgenic non-human animal is a male non-human animal. In other preferred embodiments the transgenic non-human animal is a female non-human animal. According to other embodiments, the transgenic non-human animal oocyte, blastocyst, embryo, or offspring may be used as a model for a human disease, as a model to study human disease or to screen molecules, compounds and compositions. In certain embodiments, the cells of the transgenic oocyte, zygote, blastocyst, or embryo are used to establish embryonic stem (ES) cell lines. Stem cells are defined as cells that have extensive proliferation potential, differentiate into several cell lineages, and repopulate tissues upon transplantation. (Thomson, J. et al. 1995; Thomson, J. A. et al. 1998; Shamblott, M. et al. 1998; Williams, R. L. et al. 1988; Orkin, S. 1998; Reubinoff, B. E., et al. 2000).

Diagnostic Methods

Disclosed herein are methods of diagnosing inflammation comprising, detecting the presence of soluble LIGHT in a sample from a subject. The inflammation comprises one or more of scleroderma or hepatitis. The sample may be, for example, one or more of a blood sample, a bronchioalveolar lavage sample, a sputum sample or other tissue wherein soluble LIGHT may be detected as determined by one of skill in the art. One or more of inflammation, clearance of bacteria, liver inflammation, infiltration of inflammatory cells, hepatocyte necrosis, liver cirrhosis, and/or hepatocellular carcinoma may also be used as diagnostic indicators together with level of soluble LIGHT.

Kits

The invention also provides kits useful in practicing the methods of the invention. In one embodiment, a kit of the invention includes a protein family member modulator, e.g., contained in a suitable container. Provided herein, according to one aspect, are kits comprising an modulator and a pharmaceutically acceptable carrier and b) instructions for use. In a variation of this embodiment, the protein family member modulator is formulated in a pharmaceutically acceptable carrier. The kit preferably includes instructions for administering the N-type modulator to a subject to reduce or prevent a drug-related effect or behavior.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### Example 1

### Mice, Cell Lines, and Reagents

C57BL/6J (B6) and BALB/c mice were purchased from the National Cancer Institute (Frederick, Md., USA) and Jackson Laboratory, respectively. The mice deficient in CD1d or LIGHT (B6 background) were described previously (7, 32). Age-matched 5- to 8-week-old mice were used for all experiments. All the animal experiments described in this manuscript were approved by the Johns Hopkins Animal Care and Use Committee of the Johns Hopkins University School of Medicine. A20 and 293T cells were purchased from ATCC. LIGHT-flag, LTβR-Ig, and HVEM-Ig fusion proteins were prepared as previously described (3, 5). Anti-mouse HVEM mAb (clone, LH1) and anti-mouse LTβR mAb (clone, LLTB1) were generated in our laboratory as described before (54). PK136 hybridoma was purchased from ATCC, and mAb was purified as previously described (54). Anti-asialo GM1 was purchased from Cedarlane Laboratories Ltd. Mouse IgG, rat IgG, and human IgG were purchased from Sigma-Aldrich. Hamster IgG was purchased from Rockland Immunochemicals. All cDNA plasmids were purified by an EndoFree Maxi preparation kit (QIAGEN).

### ConA-Induced Hepatitis.

The mice were injected i.v. with either a lethal dose (25-30 mg/kg) or a sublethal dose (12.5 mg/kg) of ConA (Sigma-Aldrich) in PBS with or without hydrodynamic injection of plasmid DNA. Mouse survival and serum ALT level were monitored periodically. Serum ALT level was measured by a Transaminase kit (Sigma-Aldrich) according to the manufacturer's instructions. In some experiments, livers were harvested from the treated mice, fixed in formalin solution, and embedded with paraffin. The sections were stained with H&E for histological study.

L. monocytogenes-induced Hepatitis.

The mice were infected i.p. with virulent L. monocytogenes (strain DP-L4056), which was kindly provided by Thomas W. Dubensky Jr. from Cerus Corp. Mouse survival was monitored thereafter. In some experiments, liver and spleen were harvested 3 days after infection for histological analysis and measurement of bacterial titer by plating of homogenized organs on CHROMagar Listeria plates (BD Diagnostics).

### Northern Blot Analysis.

Total RNA was extracted from liver and spleen using an RNeasy Mini Kit (Qiagen). Purified RNA (10 μg/sample) was separated by electrophoresis on a 1.5% denaturing agarose gel. The fractionated RNA was then transferred onto a Hybond-N+ membrane (Amersham Pharmacia Biotech), and blotted with 32P-labeled full-length mouse LIGHT, HVEM, LTβR, or GAPDH cDNA probe using the a Rediprime kit (Amersham Pharmacia Biotech).

### ELISA Specific to Mouse LIGHT.

Two distinct LIGHT-specific Abs, ML163 and ML209, were established as previously described (5). For LIGHT-specific ELISA, ML163 (2 μg/ml) was coated on the plate, and biotin-conjugated ML209 (5 μg/ml) was used as detection Ab. ELISA was conducted according to the procedures described previously (5). A linear standard curve was obtained with LIGHT-flag fusion protein as a positive control.

### In Vitro Gene Expression and Hydrodynamic Injection.

Lipofectamine 2000 (Invitrogen Corp.) was used for gene transfection into 293T cells according to the manufacturer's instructions. For in vivo gene expression, hydrodynamic injection of plasmids, in which 20 μg of plasmid DNA in 2 ml PBS is rapidly injected into the tail vein, was performed as previously described (27). A sublethal dose of ConA (12.5 mg/kg) was combined in the diluents in some experiments.

### Generation of LIGHT Mutants.

A plasmid encoding a single-amino-acid substitution of Phe for Tyr173 (Y173F) was generated by 2-step PCR in which wild-type mouse LIGHT cDNA was used as the template. First, overlapping oligonucleotide primers encoding the desired mutations were synthesized, and 2 flanking 5′ and 3′ primers were designed with XbaI and BamHI restriction sites, respectively. Appropriate regions of cDNA were initially amplified using corresponding overlapping and flanking primers. Then, using the flanking 5′ and 3′ primers, fragments whose sequences overlapped were fused together and amplified. PCR product was digested with XbaI and BamHI and ligated into XbaI/BamHI-digested pcDNA3.1 vectors (Invitrogen Corp.). Plasmids encoding LIGHTL mutant were designed to remove amino acids from Leu63 to Asp84 of LIGHT. LIGHTL was similarly constructed by 2-step PCR using overlapping oligonucleotide primers encoding the sequences adjacent to the desired deletion. To verify the accuracy of mutation, both mutants were sequenced using an ABI PRISM 310 Genetic Analyzer (Applied Biosystems).

### Statistics.

ALT levels and LIGHT levels in the serum were compared between groups using 2-tailed Student's t test Survival experiments are shown as Kaplan-Meier survival curves and analyzed using the log rank test. P values less than 0.05 were considered statistically significant in both tests.

Nonstandard abbreviations used: ALT, alanine aminotransferase; B6, C57BL/6J; BTLA, B and T lymphocyte attenuator; ConA, concanavalin A; HVEM, herpes virus entry mediator; LIGHT, homologous to lymphotoxin, exhibits inducible expression, and competes with HSV glycoprotein D for herpes virus entry mediator, a receptor expressed by T lymphocytes; LTβR, lymphotoxin-β receptor.

### Example 2

### Increased Expression and a Pathogenic Role for LIGHT in Experimental Hepatitis

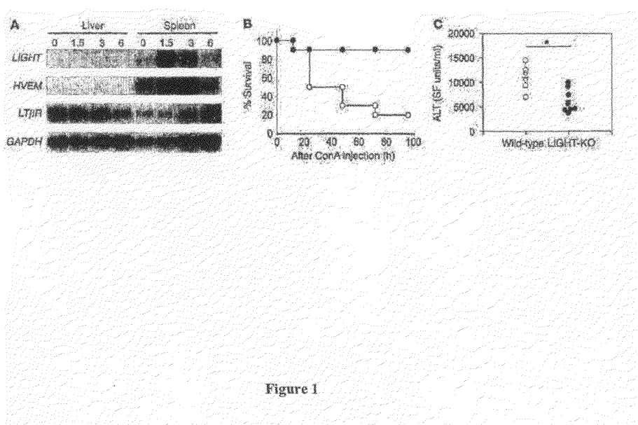

To study a potential role for LIGHT in liver inflammation, we took advantage of a mouse hepatitis model induced by concanavalin A (ConA) injection (26). First, we investigated the expression of LIGHT and its receptors in this model. Northern blot analysis showed that 1.5-3 hours after ConA treatment, LIGHT and HVEM mRNA expression was upregulated predominantly in the spleen, while LTβR was constitutively expressed in the liver, irrespective of ConA treatment (FIG. 1A). This finding indicated an enhanced expression of LIGHT in the early phase of experimental acute hepatitis, leading us to further investigate the pathogenic role of LIGHT in the liver.

Using LIGHT-deficient mice (7), we then examined whether the increase of LIGHT expression was responsible for the pathogenesis of ConA-induced hepatitis. Four days after injection of a lethal dose of ConA, 80% of control C57BL/6J (B6) mice died of acute hepatitis (FIG. 1B), which was characterized by a massive infiltration of inflammatory cells in the liver (data not shown) and an elevated serum alanine aminotransferase (ALT) (FIG. 1C). In contrast, more than 90% of LIGHT-deficient mice survived indefinitely with significantly lower levels of ALT. These results clearly indicate that LIGHT expression is an event for the initiation and/or progression of ConA-induced hepatitis.

### Proinflammatory Functions of Soluble LIGHT as a Cytokine.

To explore the mechanisms underlying LIGHT-mediated liver inflammation, we raised 3 questions to be addressed. The first is whether the soluble form of LIGHT contributes to the liver pathogenesis, since LIGHT is cleaved from the cell membrane by MMP along with inflammatory stimuli (15, 16). The second question is whether HVEM or LTβR is responsible for this effect as a functional receptor. The third one is how and which cells produce LIGHT that mediates liver inflammation.

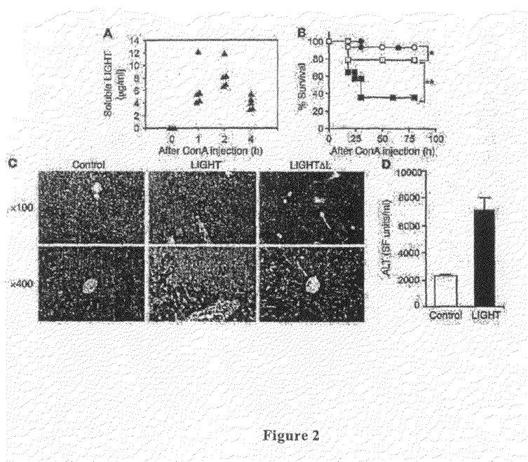

To address the first question, we examined whether the soluble form of LIGHT is detectable in vivo upon ConA treatment. By a sandwich ELISA specific to mouse LIGHT, soluble LIGHT was detected in the mouse sera as soon as 1 hour after ConA treatment, followed by a rapid decline after 4 hours (FIG. 2A), suggesting a potential role of soluble LIGHT in the pathogenesis of liver inflammation. To further ascertain this, we designed a LIGHT mutant resistant to the enzymatic cleavage. LIGHTL, a deletion mutant lacking amino acids from Leu63 to Asp84, loses the capacity to produce soluble LIGHT both in vitro upon transfection into 293T cells and in in vivo gene expression by hydrodynamic injection (27); rather, it preferentially resides on the cell membrane (Supplemental FIG. 1; supplemental material available online with this article; doi: 10.1172/JCI27083DS1). This mutant is not functionally compromised, as it can cause cell death in HT-29 cells in the presence of IFN-γ at levels similar to those required of full-length LIGHT (Supplemental FIG. 2). Together with sublethal ConA treatment, in vivo expression of wild-type LIGHT by hydrodynamic injection resulted in a high mortality of recipient mice (FIG. 2B). In contrast, the mice expressing LIGHTL survived as well as the mice receiving control vector plasmids. Consistent with these findings, pathological analyses indicated that hepatocytes were largely free from necrotic cell death after LIGHTL expression, whereas there were massive necrotic foci of hepatocytes after wild-type LIGHT expression (FIG. 2C). In addition, direct administration of recombinant soluble LIGHT protein significantly increased the serum ALT level at 6 hours (FIG. 2D) and led to the death of all the mice by 24 hours (data not shown). Collectively, these findings suggest that soluble LIGHT production by the cleavage of the membrane-bound form is a mechanism for hepatitis progression.

### Role of LTβR, But not HVEM, in the Proinflammatory Effects of LIGHT.

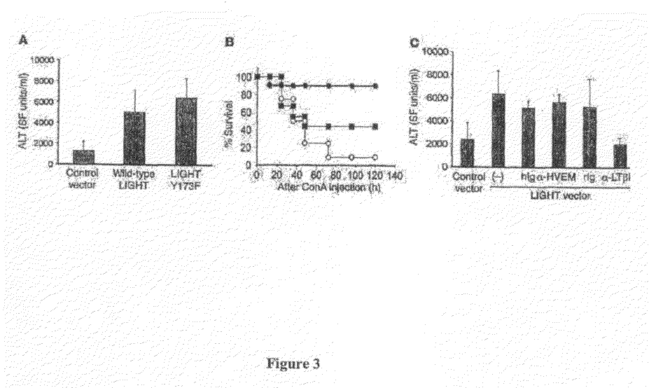

We next addressed whether HVEM or LTβR plays a responsible role in liver inflammation as a functional receptor of soluble LIGHT. To this end, we generated another mutant of mouse LIGHT, Y173F, by a single-amino-acid substitution of Phe for Tyr173. LIGHT Y173F selectively loses the binding to HVEM but not LTβR (Supplemental FIG. 3), consistent with the analogous mutant of human LIGHT (9). Upon hydrodynamic injection into mice with a sublethal dose of ConA, LIGHT Y173F mediated hepatitis at a level comparable to that of wild-type LIGHT (FIG. 3A), suggesting a dispensable role of HVEM in LIGHT-mediated hepatitis.

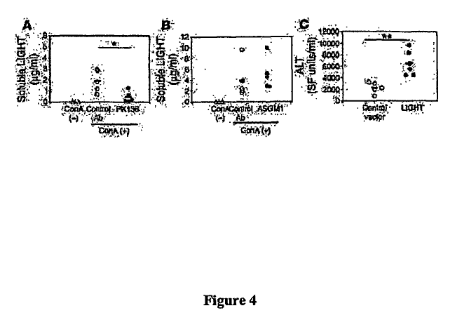

To further confirm this notion, we generated antagonistic mAbs to LTβR or HVEM. Our anti-LTβR Ab selectively blocks LIGHT-LTβR interaction but not LTβ-LTβR interaction, while anti-HVEM mAb interferes with LIGHT-HVEM interaction but not interaction between B and T lymphocyte attenuator (BTLA) and HVEM (Supplemental FIG. 4). Injection of anti-LTβR mAb profoundly decreased the mortality of mice with hepatitis induced by a lethal dose of ConA, whereas the anti-HVEM Ab showed a marginal effect on the survival (FIG. 3B). Similarly, in the hepatitis model induced by hydrodynamic injection of LIGHT with sublethal ConA treatment, blockade of LIGHT-LTβR, but not LIGHT-HVEM, resulted in a significant decrease of the serum ALT level (FIG. 3C). Taken together, these findings indicate that LIGHT-LTβR interaction is necessary and sufficient for LIGHT-mediated liver inflammation.

### Role of NK1.1+ T Cells in the Production of Soluble LIGHT in ConA-Induced Hepatitis.

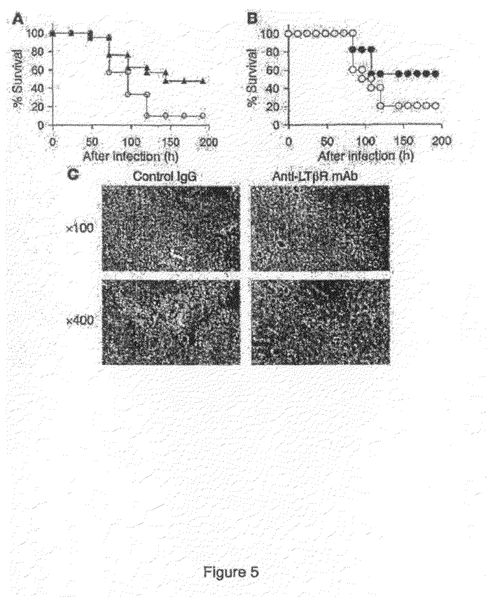

Since a role of NK1.1+ T cells (NKT cells) in the pathogenesis of ConA-induced hepatitis has been described (28, 29), we next examined a potential role of NKT cells in the production of soluble LIGHT. Mice treated with anti-NK1.1 mAb, which depletes NK and NKT cells in vivo (30, 31), expressed significantly lower amounts of soluble LIGHT than the control Ab-treated mice in response to ConA injection (FIG. 4A). In contrast, the mice treated with asialo GM1, which depletes NK but not NKT cells (29), produced as much soluble LIGHT as those treated with control Ab (FIG. 4B), suggesting a role of NKT cells in the production of soluble LIGHT. On the other hand, when LIGHT was exogenously expressed by hydrodynamic injection, CD1d-deficient mice lacking NKT cells (32) showed a significant increase of serum ALT (FIG. 4C), indicating a dispensable role of NKT cells in the effector phase of LIGHT-mediated hepatitis. In addition, given that full-length LIGHT was expressed in this experiment, NKT cells may not be required for the cleavage of membrane-bound LIGHT. In fact, soluble LIGHT was detected at normal levels in anti-NK1.1 mAb-treated mice after hydrodynamic expression of full-length LIGHT (Supplemental FIG. 5). Furthermore, exogenous expression of LIGHT mediates a certain level of hepatitis even in the absence of ConA or in mice deficient in the Rag gene (Supplemental FIG. 6), suggesting that cleavage and effector functions of soluble LIGHT require neither other inflammatory stimuli nor adaptive immune cells. Taken together, our findings indicate that NKT cells function as a cellular source of LIGHT for hepatitis, rather than a regulator of cleavage or effector cells downstream of LIGHT.

Therapeutic Potential of the LIGHT-LTβR Pathway in Listeria monocytogenes-Induced Hepatitis.

Lastly, we evaluated the pathogenic role and therapeutic potential of LIGHT in an acute hepatitis model induced by Listeria monocytogenes. Intraperitoneal infection with high-dose L. monocytogenes (2×LD50 per mouse) causes acute hepatitis associated with an immune cell infiltration and hepatocyte necrosis, ultimately leading to death in about 5 days (33). When we challenged wild-type or LIGHT-deficient B6 mice with this dose of L. monocytogenes, we found that the survival of LIGHT-deficient mice was significantly longer than that of the control mice (FIG. 5A). Similarly, selective blockade of LIGHT-LTβR interaction by our anti-LTβR mAb significantly prolonged the survival of high-dose L. monocytogenes-infected mice compared with those treated with control Ab (FIG. 5B). This effect was not due to an enhanced clearance of bacteria, since the titers of L. monocytogenes in liver and spleen were comparable between anti-LTβR mAb- and control Ab-treated mice (data not shown). In addition, we detected a low but significant amount of soluble LIGHT in the serum 24 hours after infection (100±70 ng/ml, n=5). Pathological analyses revealed that anti-LTβR mAb treatment decreased the infiltration of inflammatory cells and the necrosis of hepatocytes, thus maintaining overall integrity of liver microstructure (FIG. 5C). These findings indicate that LIGHT plays a pathological role in L. monocytogenes-induced hepatitis and that blockade of the LIGHT-LTβR pathway has a therapeutic potential in hepatic inflammation mediated by liver-tropic pathogen infections.

A novel function of LIGHT as a proinflammatory cytokine for hepatocyte damage was discovered. During the course of experimental hepatitis, activation of immune cells, predominantly NKT cells, leads to LIGHT production and subsequent cleavage as a cytokine. Soluble LIGHT circulates systemically and mediates hepatocyte death through the interaction with LTβR. Selective blockade of LIGHT-LTβR interaction ameliorates experimental hepatitis induced by ConA or L. monocytogenes, showing it as a nonredundant role of a mechanism in promoting liver inflammation.

The pathogenic effects of the role of the interaction of LIGHT with HVEM and LTβR in various inflammatory diseases and underlying mechanism have not been fully explored. However, here, using LIGHT-deficient mice, it was observed that a role of LIGHT in initiating and/or promoting inflammatory responses, rather than its being merely a by-product of the inflammatory cascade. Without wishing to be bound by any particular theories, since LIGHT-deficient mice display a phenotype of impaired T cell immunity (6-8), it is worth considering whether lymphocyte activation through LIGHT costimulatory effects contributes to the inflammatory responses. In this regard, we show herein that using anti-HVEM mAb as well as LIGHT Y173F mutant suggest that a costimulatory role through HVEM is not necessary for LIGHT-mediated liver inflammation. This notion could also be supported by evidence that LIGHT-deficient mice exhibit an immune dysfunction predominantly in CD8+ T cells (6-8), whereas CD4+ T cells and NKT cells are the central players in ConA-induced hepatitis (26, 28). In a model of L. monocytogenes-induced hepatitis, we found that the difference in survival of LIGHT-deficient mice becomes evident between days 3 and 5 after infection, when adaptive immunity including pathogen-specific CD8+ T cell responses has not yet dominated (33). In addition, selective blockade of LIGHT-LTβR with anti-LTβR mAb ameliorated hepatitis as effectively as LIGHT deficiency did, suggesting a negligible role of the HVEM costimulatory pathway. Interestingly, a recent study reported that HVEM-deficient mice have enhanced responses to ConA with severe damage of the spleen, but not the liver, and elevated levels of serum cytokines (34). These phenotypes are likely due to a lack of inhibitory signal through BTLA, another binding partner of HVEM (35). In light of these findings, it can be postulated that LIGHT mediates inflammatory responses through LTβR, but not HVEM, on nonlymphocyte populations whereas HVEM-BTLA interaction negatively regulates inflammation by attenuating lymphocyte activation.

The intracellular region of LTβR has been shown to have specific domains for binding to TRAF-2, -3, and -5, which are responsible for the delivery of death signal (39). LIGHT-LTβR interaction mediates cell death of certain tumor cell lines in the presence of IFN-γ, in which p38-MAPK and poly(ADP-ribose) polymerase (PARP) pathways induce upregulation of proapoptotic molecules such as Bak and downregulation of antiapoptotic molecules like Bcl-2 (40). In addition, the association of proapoptotic molecules like Smac and IAP-1, along with TRAF-2 and TRAF-3, with the endogenous LIGHT-LTβR complex has been reported (41). More specifically, in a hepatoma cell line, Hep3B, it has been postulated that LIGHT mediates cell death via activation of ROS and Ask-1 downstream of the TRAF-3 and TRAF-5 signals from the LTβR (42).

Although there is evidence for the presence of soluble LIGHT cleaved by MMP (15, 16), to our knowledge, no studies have characterized the biological functions of soluble LIGHT. Herein it is shown that a LIGHT mutant indicates that soluble LIGHT plays a role in the pathogenesis of liver inflammation. Given that hydrodynamic injection of plasmid leads to a predominant gene expression in hepatocytes (43), the inability of LIGHTL to mediate hepatitis suggests that a widespread distribution of soluble LIGHT, rather than cell-to-cell contact, might be useful to trigger inflammation against tightly packed cells such as hepatocytes. In addition, we detected soluble LIGHT in the serum after ConA injection (FIG. 2A) and found the ability of soluble LIGHT to mediate hepatitis (FIG. 2D), supporting a function of soluble LIGHT. Indeed, this notion is consistent with previous reports that certain MMP inhibitors protect mice from the lethal hepatitis induced by endotoxin (44) or hepatotoxin (45). In clinical situations, a recent study indicated the presence of soluble LIGHT in the bronchioalveolar lavage of scleroderma patients with active inflammation but not in those without active inflammation (46). We also found low but detectable levels of soluble LIGHT in about 30% of patients chronically infected with hepatitis B virus (K. Tamada et al., unpublished data). Taken together, these findings suggest that soluble LIGHT functions as an accelerator for tissue inflammation as well as a potential clinical marker for inflammatory activation.

There is evidence that NKT cells play an role in the pathogenesis of ConA-induced hepatitis (28, 29). It was also suggested that the infection of L. monocytogenes is associated with the activation of NKT cells (47, 48). Although the mechanism of liver damage mediated by NKT cells has not been entirely explored, their expression of FasL and IFN-γ may cause hepatocyte death directly or indirectly through the activation of Kupffer cells to produce TNF-α (49). Osteopontin derived from NKT cells and TNF-related apoptosis-inducing ligand (TRAIL) were also shown to be significant contributors to liver inflammation (50, 51). Our study here indicated that NKT cells are required for the production, but not the cleavage or effector functions, of soluble LIGHT induced by ConA injection. Interestingly, serum levels of IFN-γ and TNF-α, but not osteopontin, in response to ConA injection are significantly decreased in LIGHT-deficient mice (Supplemental FIG. 7). Although we have not explored a potential involvement of these effects in LIGHT-mediated hepatitis, it is conceivable that inflammatory mediators downstream of LIGHT contribute to the pathogenesis directly or indirectly through the synergistic effects with LIGHT (24).

Hepatic inflammation and liver regeneration appear to be 2 sides of the same coin. TNF ligand superfamily molecules that mediate hepatic death and inflammation also accelerate liver regeneration, as shown by TNF-α and FasL (52, 53). A recent study by Anders et al. indicated that the interaction of LIGHT and LTβ with LTβR also plays a role in stimulating liver regeneration (25). This dual function of LIGHT on hepatocytes could explain seemingly contradictory findings that it can either protect hepatocytes from death (36) or cause apoptosis itself (24). Thus, LIGHT as well as TNF-α and FasL has integral functions in the regulation of liver homeostasis. It seems that these factors play nonredundant roles that cannot be compensated for by others, since manipulation of any of these factors significantly changes liver homeostasis. Our work thus identifies a role for soluble LIGHT-LTβR interaction in the pathogenesis of liver inflammation. Selective regulation of this arm among interactions between LIGHT, LTβR, HVEM, and BTLA would enhance our ability to intervene in the inflammatory diseases without affecting other functions associated with these molecular pathways.

### REFERENCES

1. Aggarwal. B. B. Signalling pathways of the TNF superfamily: a double-edged sword. Nat. Rev. Immunol. 2003; 3:745-756.
2. Mauri D. N., et al. LIGHT, a new member of the TNF superfamily, and lymphotoxin alpha are ligands for herpesvirus entry mediator. Immunity. 1998; 8:21-30.
3. Tamada K., et al. LIGHT, a TNF-like molecule, costimulates T cell proliferation and is required for dendritic cell-mediated allogeneic T cell response. J. Immunol. 2000; 164:4105-4110.
4. Yu K. Y., et al. A newly identified member of tumor necrosis factor receptor superfamily (TR6) suppresses LIGHT-mediated apoptosis. J. Biol. Chem. 1999; 274:13733-13736.
5. Tamada K., et al. Modulation of T-cell-mediated immunity in tumor and graft-versus-host disease models through the LIGHT co-stimulatory pathway. Nat. Med. 2000; 6:283-289.
6. Scheu S., et al. Targeted disruption of LIGHT causes defects in costimulatory T cell activation and reveals cooperation with lymphotoxin beta in mesenteric lymph node genesis. J. Exp. Med. 2002; 195:1613-1624.
7. Tamada K., et al. Cutting edge: selective impairment of CD8+ T cell function in mice lacking the TNF superfamily member LIGHT. J. Immunol. 2002; 168:4832-4835.
8. Ye Q., et al. Modulation of LIGHT-HVEM costimulation prolongs cardiac allograft survival. J. Exp. Med. 2002; 195:795-800.
9. Rooney I. A., et al. The lymphotoxin-beta receptor is necessary and sufficient for LIGHT-mediated apoptosis of tumor cells. J. Biol. Chem. 2000; 275:14307-14315.
10. Zhai Y., et al. LIGHT, a novel ligand for lymphotoxin beta receptor and TR2/HVEM induces apoptosis and suppresses in vivo tumor formation via gene transfer. J. Clin. Invest. 1998; 102:1142-1151.
11. Lee W. H., et al. Tumor necrosis factor receptor superfamily 14 is involved in atherogenesis by inducing proinflammatory cytokines and matrix metalloproteinases. Arterioscler. Thromb. Vasc. Biol. 2001; 21:2004-2010.
12. Kim W. J., et al. LIGHT is involved in the pathogenesis of rheumatoid arthritis by inducing the expression of pro-inflammatory cytokines and MMP-9 in macrophages. Immunology. 2005; 114:272-279.
13. Wang J., et al. Dysregulated LIGHT expression on T cells mediates intestinal inflammation and contributes to IgA nephropathy. J. Clin. Invest. 2004; 113:826-835. doi: 10.1172/JCI200420096.
14. Wang J., et al. The critical role of LIGHT in promoting intestinal inflammation and Crohn's disease. J. Immunol. 2005; 174:8173-8182.
15. Granger S. W., Butrovich K. D., Houshmand P., Edwards W. R., Ware C. F. Genomic characterization of LIGHT reveals linkage to an immune response locus on chromosome 19p13.3 and distinct isoforms generated by alternate splicing or proteolysis. J. Immunol. 2001; 167:5122-5128.
16. Morel Y., et al. Reciprocal expression of the TNF family receptor herpes virus entry mediator and its ligand LIGHT on activated T cells: LIGHT down-regulates its own receptor. J. Immunol. 2000; 165:4397-4404.
17. Yin X. M., Ding W. X. Death receptor activation-induced hepatocyte apoptosis and liver injury. Curr. Mol. Med. 2003; 3:491-508.
18. Shaikh R. B., et al. Constitutive expression of LIGHT on T cells leads to lymphocyte activation, inflammation, and tissue destruction. J. Immunol. 2001; 167:6330-6337.
19. Wang J., et al. The regulation of T cell homeostasis and autoimmunity by T cell-derived LIGHT. J. Clin. Invest. 2001; 108:1771-1780. doi: 10.1172/JCI200113827.
20. Lowes K. N., Croager E. J., Abraham L. J., Olynyk J. K., Yeoh G. C. Upregulation of lymphotoxin beta expression in liver progenitor (oval) cells in chronic hepatitis C. Gut. 2003; 52:1327-1332.
21. Chen C. M., You L. R., Hwang L. H., Lee Y. H. Direct interaction of hepatitis C virus core protein with the cellular lymphotoxin-beta receptor modulates the signal pathway of the lymphotoxin-beta receptor. J. Virol. 1997; 71:9417-9426.
22. Matsumoto M., et al. Hepatitis C virus core protein interacts with the cytoplasmic tail of lymphotoxin-beta receptor. J. Virol. 1997; 71:1301-1309.
23. You L. R., Chen C. M., Lee Y. H. Hepatitis C virus core protein enhances NF-kappaB signal pathway triggering by lymphotoxin-beta receptor ligand and tumor necrosis factor alpha. J. Virol. 1999; 73:1672-1681.
24. Chen M. C., Hsu T. L., Luh T. Y., Hsieh S. L. Overexpression of bcl-2 enhances LIGHT- and interferon-gamma-mediated apoptosis in Hep3BT2 cells. J. Biol. Chem. 2000; 275:38794-38801.
25. Anders R. A., Subudhi S. K., Wang J., Pfeffer K., Fu Y. X. Contribution of the lymphotoxin beta receptor to liver regeneration. J. Immunol. 2005; 175:1295-1300.
26. Tiegs G., Hentschel J., Wendel A. A T cell-dependent experimental liver injury in mice inducible by concanavalin A. J. Clin. Invest. 1992; 90:196-203.
27. Knapp J. E., Liu D. Hydrodynamic delivery of DNA. Methods Mol. Biol. 2004; 245:245-250.
28. Takeda K., et al. Critical contribution of liver natural killer T cells to a murine model of hepatitis. Proc. Natl. Acad. Sci. U.S.A. 2000; 97:5498-5503.
29. Toyabe S., et al. Requirement of IL-4 and liver NK1+ T cells for concanavalin A-induced hepatic injury in mice. J. Immunol. 1997; 159:1537-1542.
30. Seaman W. E., Sleisenger M., Eriksson E., Koo G. C. Depletion of natural killer cells in mice by monoclonal antibody to NK-1.1. Reduction in host defense against malignancy without loss of cellular or humoral immunity. J. Immunol. 1987; 138:4539-4544.
31. von der Weid T., Beebe A. M., Roopenian D. C., Coffman R. L. Early production of IL-4 and induction of Th2 responses in the lymph node originate from an MHC class I-independent CD4+NK1.1-T cell population. J. Immunol. 1996; 157:4421-4427.
32. Chen Y. H., Chiu N. M., Mandal M., Wang N., Wang C. R. Impaired NK1+T cell development and early IL-4 production in CD1-deficient mice. Immunity. 1997; 6:459-467.
33. Pamer E. G. Immune responses to Listeria monocytogenes. Nat. Rev. Immunol. 2004; 4:812-823.
34. Wang Y., et al. The role of herpesvirus entry mediator as a negative regulator of T cell-mediated responses. J. Clin. Invest. 2005; 115:711-717. doi: 10.1172/JCI200522982.
35. Sedy J. R., et al. B and T lymphocyte attenuator regulates T cell activation through interaction with herpesvirus entry mediator. Nat. Immunol. 2005; 6:90-98.
36. Matsui H., Hikichi Y., Tsuji I., Yamada T., Shintani Y. LIGHT, a member of the tumor necrosis factor ligand superfamily, prevents tumor necrosis factor-alpha-mediated human primary hepatocyte apoptosis, but not Fas-mediated apoptosis. J. Biol. Chem. 2002; 277:50054-50061.
37. Browning J. L., et al. Characterization of lymphotoxin-alpha beta complexes on the surface of mouse lymphocytes. J. Immunol. 1997; 159:3288-3298.
38. Schneider K., Potter K. G., Ware C. F. Lymphotoxin and LIGHT signaling pathways and target genes. Immunol. Rev. 2004; 202:49-66.
39. Force W. R., et al. Discrete signaling regions in the lymphotoxin-beta receptor for tumor necrosis factor receptor-associated factor binding, subcellular localization, and activation of cell death and NF-kappaB pathways. J. Biol. Chem. 2000; 275:11121-11129.
40. Chang Y. H., Chao Y., Hsieh S. L., Lin W. W. Mechanism of LIGHT/interferon-gamma-induced cell death in HT-29 cells. J. Cell. Biochem. 2004; 93:1188-1202.
41. Kuai J., et al. Endogenous association of TRAF2, TRAF3, cIAP1, and Smac with lymphotoxin beta receptor reveals a novel mechanism of apoptosis. J. Biol. Chem. 2003; 278:14363-14369.
42. Chen M. C., et al. The role of apoptosis signal-regulating kinase 1 in lymphotoxin-beta receptor-mediated cell death. J. Biol. Chem. 2003; 278:16073-16081.
43. Kobayashi N., Kuramoto T., Yamaoka K., Hashida M., Takakura Y. Hepatic uptake and gene expression mechanisms following intravenous administration of plasmid DNA by conventional and hydrodynamics-based procedures. J. Pharmacol. Exp. Ther. 2001; 297:853-860.
44. Mohler K. M., et al. Protection against a lethal dose of endotoxin by an inhibitor of tumour necrosis factor processing. Nature. 1994; 370:218-220.
45. Wielockx B., et al. Inhibition of matrix metalloproteinases blocks lethal hepatitis and apoptosis induced by tumor necrosis factor and allows safe antitumor therapy. Nat. Med. 2001; 7:1202-1208.
46. Luzina I. G., et al. Occurrence of an activated, profibrotic pattern of gene expression in lung CD8+ T cells from scleroderma patients. Arthritis Rheum. 2003; 48:2262-2274.
47. Arrunategui-Correa V., Lenz L., Kim H. S. CD1d-independent regulation of NKT cell migration and cytokine production upon Listeria monocytogenes infection. Cell Immunol. 2004; 232:38-48.
48. Ranson T., et al. Invariant V alpha 14+ NKT cells participate in the early response to enteric Listeria monocytogenes infection. J. Immunol. 2005; 175:1137-1144.
49. Exley M. A., Koziel M. J. To be or not to be NKT: natural killer T cells in the liver. Hepatology. 2004; 40:1033-1040.
50. Diao H., et al. Osteopontin as a mediator of NKT cell function in T cell-mediated liver diseases. Immunity. 2004; 21:539-550.
51. Zheng S. J., Wang P., Tsabary G., Chen Y. H. Critical roles of TRAIL in hepatic cell death and hepatic inflammation. J. Clin. Invest. 2004; 113:58-64. doi: 10.1172/JCI200419255.
52. Desbarats J., Newell M. K. Fas engagement accelerates liver regeneration after partial hepatectomy. Nat. Med. 2000; 6:920-923.
53. Yamada Y., Kirillova I., Peschon J. J., Fausto N. Initiation of liver growth by tumor necrosis factor: deficient liver regeneration in mice lacking type I tumor necrosis factor receptor. Proc. Natl. Acad. Sci. U.S.A. 1997; 94:1441-1446.
54. Wilcox R. A., et al. Provision of antigen and CD137 signaling breaks immunological ignorance, promoting regression of poorly immunogenic tumors. J. Clin. Invest. 2002; 109:651-659. doi: 10.1172/JCI2002

1. A method of modulating an interaction between LIGHT and LTβR comprising, administering a LIGHT pathway protein modulator.

2. A method of modulating inflammation in a mammal, comprising modulating the LIGHT pathway protein signaling wherein disrupting an interaction between LIGHT and LTβR modulates inflammation of the a cell.

3. A method for the treatment and/or prophylaxis of a condition characterized by aberrant or otherwise unwanted inflammation in a subject, comprising modulating the LIGHT pathway protein signaling wherein disrupting an interaction between LIGHT and LTβR modulates inflammation of the a cell.

4. The method according to claim 1, wherein the cell is one or more of a liver cell, a cartilage cell, a digestive tract cell, neuronal cell, a pancreatic cell, a lung cell, bone tissue cell, a spleen cell, heart cell, kidney cell, a testis cell, or an intestinal tract cell.

5. The method of claim 1, wherein the protein comprises one or more LIGHT pathway family proteins.

6. The method of claim 1, wherein the LIGHT pathway family protein comprises LIGHT or LTβR.

7-13. (canceled)

14. A pharmaceutical composition comprising a pharmaceutically effective amount of a LIGHT pathway modulator effective to treat, prevent, ameliorate, reduce or alleviate a LIGHT pathway related disorder or symptoms thereof and a pharmaceutically acceptable excipient.

15. The pharmaceutical composition of claim 14, wherein the LIGHT pathway modulator is selected from one or more of a small molecule, an anti-LIGHT pathway antibody, an antigen-binding fragment of an anti-LIGHT pathway antibody, a polypeptide, a peptidomimetic, a nucleic acid encoding a peptide, or an organic molecule.

16. (canceled)

17. A method to treat, prevent, ameliorate, reduce or alleviate a LIGHT pathway related disorder or symptoms thereof, comprising: administering to a subject in need thereof a composition comprising a pharmaceutically effective amount of a LIGHT pathway modulator.

18. The method of claim 17, wherein the LIGHT pathway related disorder comprises one or more of liver inflammation, hepatitis (autoimmune and pathogen induced), arthrosclerosis, arthritis, IgA nephropathy, inflammatory bowel disease, liver cirrhosis, and hepocellular carcinoma.

19-28. (canceled)

29. A method for identifying lead compounds for a pharmacological agent useful in the treatment of a LIGHT pathway related disorder comprising:
contacting a cell stimulated with concanavalin A with a test compound, and
measuring LIGHT pathway activation or inflammation.

30-32. (canceled)

33. A kit comprising: a) a LIGHT pathway modulator and a pharmaceutically acceptable carrier and b) instructions for use.

34. A transgenic non-human animal comprising (i) a Y173F LIGHT protein or a fragment or variant thereof or (ii) a LIGHTΔL protein or a fragment or variant thereof.

35-42. (canceled)

43. A method of treating inflammation in a mammal, comprising: (a) identifying a mammal with, or at risk of developing, inflammation, wherein the cells of the cancer are identified as expressing soluble LIGHT; and (b) administering to the subject a compound comprising an agent that interferes with an interaction between LIGHT and a LTβR.

44. A method of diagnosing inflammation comprising, detecting the presence of soluble LIGHT in a sample from a subject.

45-49. (canceled)
